(19) United States
(12) Patent Application Publication
(10) Pub. No.: US 2009/0017112 A1
(43) Pub. Date: Jan. 15, 2009

(54) Compounds for Inhibiting Beta-Amyloid Production

(75) Inventors: Michael J. Mullan, Tampa, FL (US); Daniel Paris, Sarasota, FL (US); Pancham Bakshi, Sarasota, FL (US)

Correspondence Address: KING & SPALDING, 1185 AVENUE OF THE AMERICAS, NEW YORK, NY, 10036-4003, US

(73) Assignee: ROSKAMP RESEARCH LLC, Sarasota, FL (US)

(21) Appl. No.: 12/278,341

(22) PCT Filed: Feb. 28, 2007
(86) PCT No.: PCT/US07/62959

§ 371 Date: Aug. 27, 2008

Related U.S. Application Data
(60) Provisional application No. 60/777,772, filed on Mar. 1, 2006.

(51) Int. Cl. C07D 487/04; A61K 31/437; C07D 217/22; C07D 403/12; A61K 9/48; A61P 25/28; A61K 31/496; A61K 31/47

(52) U.S. Cl. 424/456; 514/299; 514/309; 546/183; 546/141; 544/362; 514/253.04

(57) ABSTRACT

Provided are compounds useful for treating diseases associated with a cerebral accumulation of Alzheimer's amyloid, such as Alzheimer's disease. Also provided are methods of treating or reducing the risk of developing β-amyloid production, β-amyloid deposition, β-amyloid neurotoxicity (including abnormal hyperphosphorylation of tau) and microgliosis associated with cerebral accumulation of Alzheimer's amyloid by administering therapeutically effective amounts of the compounds. Further provided are methods for diagnosing diseases associated with cerebral accumulation of Alzheimer's amyloid in animals or humans by administering diagnostically effective amounts of the compounds.

This application claims the benefit of priority to U.S. provisional patent application 60/777,772, filed Mar. 1, 2006, which is hereby incorporated by reference in its entirety.

### FIELD OF THE INVENTION

The present invention relates to compounds for the treatment of diseases associated with cerebral accumulation of Alzheimer's amyloid, such as Alzheimer's disease, and methods of use of the compounds for the treatment and diagnosis of diseases associated with cerebral accumulation of Alzheimer's amyloid.

### DESCRIPTION OF RELATED ART

Alzheimer's disease (AD) is the most common neurodegenerative disorder of aging, afflicting approximately 1% of the population over the age of 65. Characteristic features of the disease include neurofibrillary tangles composed of abnormal tau protein, paired helical filaments, neuronal loss, and alteration in multiple neurotransmitter systems. The hyperphosphorylation of microtubule-associated tau protein is a known marker of the pathogenic neuronal pre-tangle stage in AD brain (Tan et al., “Microglial Activation Resulting from CD40R/CD40L Interaction after Beta-Amyloid Stimulation,” Science (1999) 286:2352-55).

A significant pathological feature of AD is an overabundance of diffuse and compact senile plaques in association with limbic areas of the brain. Although these plaques contain multiple proteins, their cores are composed primarily of β-amyloid protein, a 39-43 amino acid proteolytic fragment that is proteolytically derived from amyloid precursor protein (APP), a transmembrane glycoprotein. Additionally, C-terminal fragments (CTF) of APP are known to accumulate intraneuronally in AD.

β-amyloid is derived from APP, a single-transmembrane protein with a 590 to 680 amino acid extracellular amino terminal domain and an approximately 55 amino acid cytoplasmic tail. Messenger RNA from the APP gene on chromosome 21 undergoes alternative splicing to yield eight possible isoforms, three of which (the 695, 751 and 770 amino acid isoforms) predominate in the brain. APP undergoes proteolytic processing via three enzymatic activities, termed α-, β- and γ-secretase. Alpha-secretase cleaves APP at amino acid 17 of the β-amyloid domain, thus releasing the large soluble amino-terminal fragment α-APP for secretion. Because α-secretase cleaves within the β-amyloid domain, this cleavage precludes β-amyloid formation. Alternatively, APP can be cleaved by β-secretase to define the amino terminus of β-amyloid and to generate the soluble amino-terminal fragment β-APP. Subsequent cleavage of the intracellular carboxy-terminal domain of APP by γ-secretase results in the generation of multiple peptides, the two most common being a 40 amino acid β-amyloid (Aβ1-40) and 42 amino acid β-amyloid (Aβ1-42). Aβ1-40 comprises 90-95% of the secreted β-amyloid and is the predominant species recovered from cerebrospinal fluid (Seubert et al., Nature, 359:325-7, 1992). In contrast, less than 10% of secreted β-amyloid is Aβ1-42. Despite the relative paucity of Aβ1-42 production, Aβ1-42 is the predominant species found in plaques and is deposited initially, perhaps due to its ability to form insoluble amyloid aggregates more rapidly than Aβ1-40 (Jarrett et al., Biochemistry, 32:4693-7, 1993). The abnormal accumulation of β-amyloid in the brain is believed to be due to decreased clearance of β-amyloid from the brain to the periphery or excessive production of β-amyloid. Various studies suggests excessive production of β-amyloid is due to either overexpression of APP or altered processing of APP, or mutation in the γ secretases or APP responsible for β-amyloid formation.

β-Amyloid peptides are thus believed to play a critical role in the pathobiology of AD, as all the mutations associated with the familial form of AD result in altered processing of these peptides from APP. Indeed, deposits of insoluble, or aggregated, fibrils of β-amyloid in the brain are a prominent neuropathological feature of all forms of AD, regardless of the genetic predisposition of the subject. It also has been suggested that AD pathogenesis is due to the neurotoxic properties of β-amyloid. The cytotoxicity of β-amyloid was first established in primary cell cultures from rodent brains and also in human cell cultures. The work of Mattson et al. (J. Neurosci., 12:376-389, 1992) indicates that β-amyloid, in the presence of the excitatory neurotransmitter glutamate, causes an immediate pathological increase in intracellular calcium, which is believed to be very toxic to the cell through its greatly increased second messenger activities.

Concomitant with β-amyloid production and β-amyloid deposition, there exists robust activation of inflammatory pathways in AD brain, including production of pro-inflammatory cytokines and acute-phase reactants in and around β-amyloid deposits (McGeer et al., J. Leukocyte Biol., 65:409-15, 1999). Activation of the brain's resident innate immune cells, the microglia, is thought to be intimately involved in this inflammatory cascade. It has been demonstrated that reactive microglia produce pro-inflammatory cytokines, such as inflammatory proteins and acute phase reactants, such as alpha-1-antichymotrypsin, transforming growth factor β, apolipoprotein E and complement factors, all of which have been shown to be localized to β-amyloid plaques and to promote β-amyloid plaque “condensation” or maturation (Nilsson et al., J. Neurosci. 21:1444-5, 2001), and which at high levels promote neurodegeneration. Epidemiological studies have shown that patients using non-steroidal anti-inflammatory drugs (NSAIDS) have as much as a 50% reduced risk for AD (Rogers et al., Neurobiol. Aging 17:681-6, 1996), and post-mortem evaluation of AD patients who have undergone NSAID treatment has demonstrated that risk reduction is associated with diminished numbers of activated microglia (Mackenzie et al., Neurology 50:986-90, 1998). Further, when Tg APPsw mice, a mouse model for Alzheimer's disease, are given an NSAID (ibuprofen), these animals show reduction in β-amyloid deposits, astrocytosis, and dystrophic neurites correlating with decreased microglial activation (Lim et al., J. Neurosci. 20:5709-14, 2000).

At present, treatment for AD is limited. However, there are several drugs approved by the FDA to improve or stabilize symptoms of AD (Alzheimer's Disease Medications Fact Sheet: (July 2004) U.S. Department of Health and Human Services), including Aricept® (donepezil), Exelon® (rivastigmine), Reminyl® (galantamine) Cognex® (tacrine) and Namenda® (memantine). The effects with many drugs currently in use is small (Tariot et al., JAMA (2004), 291: 317-24). Treatments for AD remain a largely unmet clinical need.

U.S. Patent Application No. 2005009885 (Jan. 13, 2005) (Mullan et al.) discloses a method for reducing beta-amyloid deposition using nilvadipine, as wells as methods of diagnosing cerebral amyloidogenic diseases using nilvadipine. Nimodipine has been studied for the treatment of dementia. Fritze et al., J. Neural Transm. (1995) 46: 439-453; and Forette et al. Lancet (1998) 352: 1347-1351).

Augmentation of capacitative calcium entry (CCE) through the identification of agonist of plasma membrane store-operated calcium channels that mediate CCE, has been suggested as a treatment for AD (Tanzi et al. Neuron (2000) 27: 561-572). U.S. Patent Application Publication No. 20020015941 (Feb. 7, 2002) discloses a method for the treatment of a neurodegenerative disease such as AD involving administering an agent which is capable of potentiating CCE.

There continues to be a need to identify compounds that can treat the inexorable progression of brain degeneration which is a hallmark of AD, wherein the treatment addresses β-amyloid production and the concomitant β-amyloid deposition, β-amyloid neurotoxicity (including abnormal hyperphosphorylation of tau), microglial-activated inflammation, and altered or over expression of APP which is seen in AD patients.

### SUMMARY

Provided are compounds that are useful in methods for the treatment of diseases associated with the accumulation of Alzheimer's amyloid.

In one embodiment, provided is a compound of Formula I or a salt, ester or prodrug thereof:

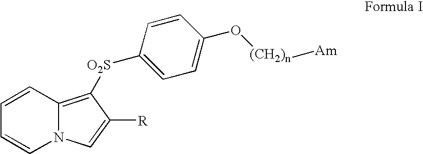

or a compound of Formula II or a salt, ester or prodrug thereof:

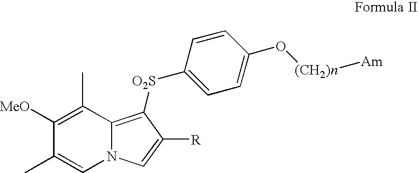

wherein:

n is 3, 4 or 5;

R is optionally substituted alkyl, e.g., methyl, ethyl, isopropyl, butyl, isobutyl; optionally substituted alkenyl; optionally substituted alkynyl; optionally substituted cycloalkyl; optionally substituted heteroalkyl; optionally substituted aryl; optionally substituted heteroaryl; or optionally substituted acyl; and

Am is the residue of an amine, such as an optionally substituted alkylamine.

In one embodiment, compounds provided herein include the following:

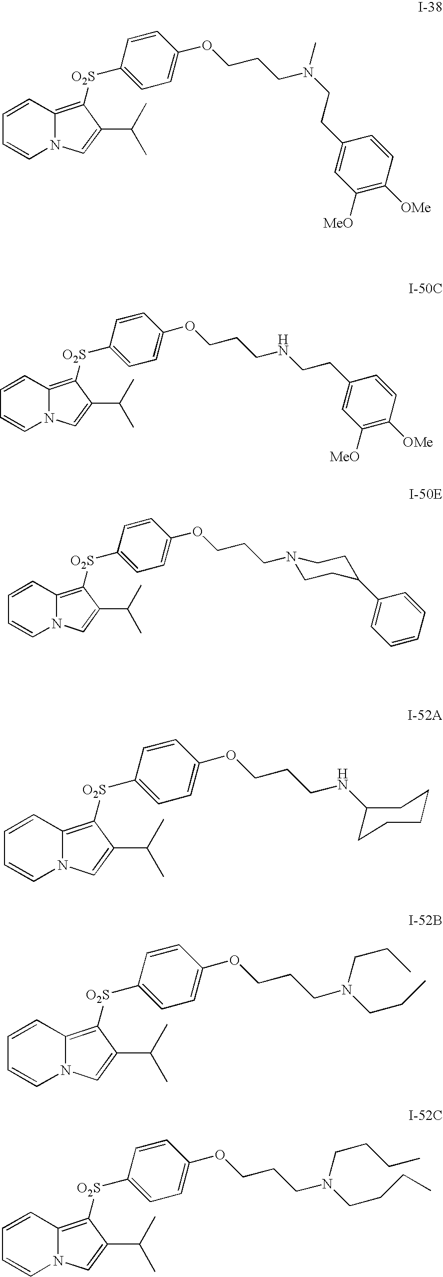

Also provided are methods of treating a disease associated with cerebral accumulation of β-amyloid in animals or humans afflicted with the disease, such as AD, by administering a therapeutically effective amount of at least one compound disclosed herein, such as a compound of Formula I, II, III or IV as disclosed herein.

The method may in one embodiment include one or more of reducing β-amyloid production, β-amyloid deposition, β-amyloid neurotoxicity (including abnormal hyperphosphorylation of tau) and microgliosis. Because most diseases having cerebral accumulation of Alzheimer's amyloid, such as AD, are chronic, progressive, intractable brain dementias, it is contemplated that the duration of treatment with at least one of the active agents can optionally last for up to the lifetime of the animal or human.

In another embodiment, a method is provided for treating a disease associated with cerebral accumulation of Alzheimer's amyloid, comprising administering to the animal or human a therapeutically effective amount of at least one compound disclosed herein, or a salt, ester or prodrug thereof. Preferably the active agent opposes the pathophysiological effects of the cerebral accumulation of Alzheimer's amyloid, and may, for example, reduce β-amyloid production, β-amyloid deposition, β-amyloid neurotoxicity and/or microgliosis in animals and humans afflicted with the disease.

In another embodiment, a diagnostic method for a disease associated with cerebral accumulation of Alzheimer's amyloid in an animal or human is provided, comprising: taking a first measurement of plasma, urine, serum, whole blood, or cerebral spinal fluid (CSF) concentration of β-amyloid in the peripheral circulation of the animal or human; administering a diagnostically effective amount in unit dosage form of at least one active compound disclosed herein, or salt, prodrug or derivative thereof, to the animal or human; taking a second measurement of plasma, serum, whole blood, urine or CSF concentration of β-amyloid in the peripheral circulation of the animal or human; and calculating the difference between the first measurement and the second measurement, wherein a change in the plasma, serum, whole blood, urine or CSF concentration of β-amyloid in the second measurement compared to the first measurement, such as an increase or decrease in concentration, indicates a possible diagnosis of a disease associated with cerebral accumulation of Alzheimer's amyloid in the animal or human.

In a further embodiment, a method is provided for treating traumatic brain injury, comprising administering to the animal or human a therapeutically effective amount in unit dosage form of at least one compound disclosed herein, or salt, ester or prodrug thereof. In one embodiment, the administration of the active agent begins immediately following the injury. In one embodiment, the compound reduces the risk of β-amyloid production, Aβ deposition, β-amyloid neurotoxicity and/or microgliosis.

The compound optionally is one that optionally reduces β-amyloid production by at least about 5%, 10%, 15%, 20%, 25%, 30%, or 50%, in cells that overexpress APP or a fragment thereof, as measured, for example in a culture medium comprising the cells.

In one embodiment, the compound optionally reduces β-amyloid production, e.g., production of total Aβ₁₋₄₀ and Aβ₁₋₄₂, by at least about 20% or more in cells that overexpress APP or a fragment thereof.

Optionally, the cells are Chinese hamster ovary cells that overexpress APP751, or are selected from human neuronal precursor cells (HNPC); primary culture of human astrocytes; neuroblastoma cells; human brain microvascular endothelial primary culture; or human umbilical cord endothelial cells (HUVEC).

In one embodiment, the compound is administered in an amount of about 0.02 to 1000 mg per unit dose; or about 0.5 to 500 mg per unit dose. Optionally pharmaceutical formulations comprising the compounds are provided that contain, e.g. 7-3000 mg, or, e.g. 10-1000 mg, or 100-500 mg of active compound.

The therapeutically effective amount of compound that is administered, e.g., in unit dosage form to animals or humans afflicted with a cerebral amyloidogenic disease or suffering from a traumatic brain injury, as well as administered for the purpose of determining the risk of developing and/or a diagnosis of a cerebral amyloidogenic disease in an animal or human, can range from for example from about 0.05 mg to 20 mg per day, about 2 mg to 15 mg per day about 4 mg to 12 mg per day, or about 8 mg per day. The daily dosage in one embodiment can be administered in a single unit dose or divided into two, three or four unit doses per day.

The disease associated with cerebral accumulation of Alzheimer's amyloid is for example, Alzheimer's disease, cerebral amyloid angiopathy, hereditary cerebral hemorrhage with amyloidosis Dutch-type, other forms of familial Alzheimer's disease and familial cerebral Alzheimer's amyloid angiopathy. Cerebral amyloidogenic diseases that can be treated or diagnosed include transmissible spongiform encephalopathy, scrapie, traumatic brain injury, cerebral amyloid angiopathy, and Gerstmann-Straussler-Scheinker syndrome.

### BRIEF DESCRIPTION OF THE DRAWINGS

FIG. 1 is a bar graph showing the effect of compounds 1-38, 1-50C and 1-36 on Aβ1-40 production by 7W WT APP 751 Chinese hamster ovary (7W WT APP 751 CHO) cells.

FIG. 2 is a bar graph showing the effect of 10 compounds at different concentrations on Aβ1-40 production by 7W WT APP751 CHO cells.

FIG. 3 is a graph showing the effect of various compounds on Aβ1-40 production by 7W WT APP751 CHO cells at various concentrations.

FIG. 4 is a bar graph showing the effect of various compounds on Aβ1-42 production by 7W WT APP751 CHO cells at various concentrations.

FIG. 5 is a bar graph showing the effect of various compounds on Aβ1-40 production by 7W WT APP751 CHO cells at various concentrations.

FIG. 6 is a bar graph showing plasma beta-amyloid (Aβ) (% of control) levels after treatment with compounds 1-52C and 1-76D in an in vivo experiment.

FIG. 7 is a bar graph showing brain water soluble beta-amyloid (Aβ) levels after treatment with compounds 1-52C and 1-76D in an in vivo experiment.

### DETAILED DESCRIPTION

Provided are compounds that can decrease β-amyloid production in mammalian cells and can be used in the diagnosis and treatment of diseases associated with the accumulation of β-amyloid in individuals. Compounds and pharmaceutical compositions comprising the compounds are provided that can be used in one embodiment to treat the inexorable progression of brain degeneration that is a hallmark of certain diseases associated with cerebral accumulation of Alzheimer's amyloid, such as Alzheimer's disease (AD), in animals and humans.

### DEFINITIONS

As used herein, the term “Alzheimer's amyloid” is defined as a β-amyloid amino acid fragment that is for example proteolytically derived from amyloid precursor protein (APP). A β-amyloid amino acid fragment may include, for example, about 5 to 43 or 5 to 47 consecutive amino acids of the β-amyloid sequence. As used herein, the terms “(β-amyloid,” “β-amyloid protein” and “Aβ” are used interchangeably with Alzheimer's amyloid that accumulates cerebrally in an animal or human.

As used herein the phrase a cell that “overexpresses APP or fragment thereof” refers to a cell that overexpresses an amyloid precursor protein, or fragment thereof, that in one preferred embodiment, includes a β-amyloid sequence and β and γ secretase cleavage sites. The cell that overexpresses APP or a fragment thereof preferably expresses an APP or fragment thereof that produces β-amyloid in the cell in which it is expressed.

As used herein, the term “amyloidogenic disease” includes a disease associated with cerebral accumulation of Alzheimer's amyloid.

The term “alkyl”, as used herein, unless otherwise specified, includes a saturated straight, branched, or cyclic, primary, secondary, or tertiary hydrocarbon, of C₁₋₂₂ and specifically includes methyl, ethyl, propyl, isopropyl, cyclopropyl, butyl, isobutyl, secbutyl, t-butyl, pentyl, cyclopentyl, isopentyl, neopentyl, hexyl, isohexyl, cyclohexyl, cyclohexylmethyl, heptyl, cycloheptyl, octyl, cyclo-octyl, dodecyl, tridecyl, pentadecyl, icosyl, hemicosyl, and decosyl. Where the alkyl group is referenced as being optionally substituted, then the group is optionally substituted with, e.g., halogen (fluoro, chloro, bromo or iodo), hydroxy, amino, alkylamino, arylamino, alkoxy, aryloxy, nitro, cyano, sulfonic acid, sulfate, heterocycle, phenyl, aryl, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl”, as used herein, and unless otherwise specified, includes a C₁ to C₄ saturated straight, branched, or if appropriate, a cyclic (for example, cyclopropyl) alkyl group.

The term “aralkyl” as used herein unless otherwise specified, includes an aryl group linked to the molecule through an alkyl group.

The term “alkaryl” as used herein unless otherwise specified, includes an alkyl group linked to the molecule through an aryl group.

The term “aryl ether” as herein unless otherwise specified, includes an aryl group linked to the molecule through an ether group.

The term “alkyl ether” as herein unless otherwise specified, includes an alkyl group linked to the molecule through an ether group.

The term “aryl thioether” as herein unless otherwise specified, includes an aryl group linked to the molecule through a sulfur.

The term “alkyl thioether” as herein unless otherwise specified, includes an alkyl group linked to the molecule through a sulfur.

The term “amino” includes an “—N(R′)₂” group, and includes primary amines, and secondary and tertiary amines which is optionally substituted for example with alkyl, aryl, heterocycle, and or sulfonyl groups. Thus, (R′)₂ may include, but is not limited to, two hydrogens, a hydrogen and an alkyl, a hydrogen and an aryl, a hydrogen and an alkenyl, two alkyls, two aryls, two alkenyls, one alkyl and one alkenyl, one alkyl and one aryl, or one aryl and one alkenyl.

Whenever a range of carbon atoms is referred to, it includes independently and separately every member of the range. As a nonlimiting example, the term “C₁-C₁₀ alkyl” is considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities.

The term “amido” includes a moiety represented by the structure “—C(O)N(R′)₂”, wherein R′ may independently include H, alkyl, alkenyl and aryl that is optionally substituted.

The term “protected” as used herein and unless otherwise defined includes a group that is added to an atom such as an oxygen, nitrogen, or phosphorus atom to prevent its further reaction or for other purposes. A wide variety of oxygen and nitrogen protecting groups are known to those skilled in the art of organic synthesis.

The term “aryl”, as used herein, and unless otherwise specified, includes a stable monocyclic, bicyclic, or tricyclic carbon ring with up to 8 members in each ring, and at least one ring being aromatic. Examples include, but are not limited to, benzyl, phenyl, biphenyl, or naphthyl. Where referenced as being optionally substituted, the aryl group can be substituted with one or more moieties including halogen (fluoro, chloro, bromo or iodo), hydroxy, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “halo”, as used herein, includes chloro, bromo, iodo, and fluoro.

The term “alkenyl” includes a straight, branched, or cyclic hydrocarbon of C2-22 with at least one double bond. Examples include, but are not limited to, vinyl, allyl, and methyl-vinyl. Where indicated as being optionally substituted, the alkenyl group can be optionally substituted in the same manner as described above for the alkyl groups.

The term “alkynyl” includes a C2-22 straight or branched hydrocarbon with at least one triple bond. Where indicated as being optionally substituted, the alkynyl group can be optionally substituted in the same manner as described above for the alkyl groups.

The term “alkoxy” includes a moiety of the structure —O-alkyl.

The term “heterocycle” or “heterocyclic” includes a saturated, unsaturated, or aromatic stable 5 to 7 membered monocyclic or 8 to 11 membered bicyclic heterocyclic ring that consists of carbon atoms and from one to three heteroatoms including but not limited to O, S, N, and P; and wherein the nitrogen and sulfur heteroatoms may optionally be oxidized, and/or the nitrogen atoms quarternized and including any bicyclic group in which any of the above-defined heterocyclic rings is fused to a benzene ring. The heterocyclic ring may be attached at any heteroatom or carbon atom which results in the creation of a stable structure. Nonlimiting examples or heterocyclic groups include pyrrolyl, pyrimidyl, pyridinyl, imidazolyl, pyridyl, furanyl, pyrazole, oxazolyl, oxirane, isooxazolyl, indolyl, isoindolyl, thiazolyl, isothiazolyl, quinolyl, tetrazolyl, bonzofuranyl, thiophrene, piperazine, and pyrrolidine.

The term “acyl” includes a group of the formula R′C(O), wherein R′ is a H, or a straight, branched, or cyclic, substituted or unsubstituted alkyl or aryl.

The term “host”, as used herein, unless otherwise specified, includes mammals (e.g., cats, dogs, horses, mice, etc.), humans, or other organisms in need of treatment, all of which can be treated or diagnosed using the methods described herein.

The term “treatment” as used herein includes any manner in which one or more of the symptoms of a disease or disorder are ameliorated or otherwise beneficially altered.

The term “pharmaceutically acceptable salt” as used herein, unless otherwise specified, includes salts of active compounds which are prepared with relatively nontoxic acids. When compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, oxalic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). The neutral forms of the compounds may be regenerated by contacting the salt with a base or acid and isolating the parent compound in the conventional manner. The pharmaceutically acceptable salt in one embodiment is a salt that is, within the scope of sound medical judgment, suitable for use in contact with the tissues of a host without undue toxicity, irritation, allergic response and the like, and is commensurate with a reasonable benefit/risk ratio and effective for their intended use.

The term “pharmaceutically acceptable esters” as used herein, unless otherwise specified, includes those esters of one or more compounds, which are, within the scope of sound medical judgment, suitable for use in contact with the tissues of hosts without undue toxicity, irritation, allergic response and the like, are commensurate with a reasonable benefit/risk ratio, and are effective for their intended use.

The term “pharmaceutically acceptable prodrugs” as used herein, unless otherwise specified, includes those prodrugs of one or more compounds of the composition which are, with the scope of sound medical judgment, suitable for use in contact with the tissues of hosts without undue toxicity, irritation, allergic response and the like, are commensurate with a reasonable benefit/risk ratio, and are effective for their intended use. Pharmaceutically acceptable prodrugs also include zwitterionic forms, where possible, of one or more compounds of the composition. The term “prodrug” includes compounds that are rapidly transformed in vivo to yield the parent compound, for example by hydrolysis in blood.

The term “enantiomerically enriched”, as used herein, refers to a compound that is a mixture of enantiomers in which one enantiomer is present in excess, and preferably present to the extent of 95% or more, and more preferably 98% or more, including 100%.

The term “optionally substituted,” as used herein, includes substituted and unsubstituted. Wherein a group is referenced as “optionally substituted” the group may be optionally substituted with e.g., halogen, hydroxyl, amino, alkylester, arylester, silylester, alkylamino, arylamino, alkylamido, arylamido, alkoxy, aryloxy, nitro, cyano, alkenyl, alkynyl, heterocycles, sulfonic acid, sulfate, phosphonic acid, phosphate, boronic acid, or borate.

### Compounds

A variety of compounds are provided as disclosed herein and below, which in one embodiment can be used in methods described herein, including the treatment or diagnosis of diseases associated with cerebral accumulation of Alzheimer's amyloid.

Exemplary compounds include a compound of Formula I or a pharmaceutically acceptable salt, ester or prodrug thereof:

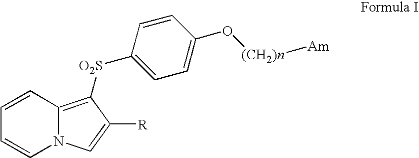

or a compound of Formula II or a pharmaceutically acceptable salt, ester or prodrug thereof:

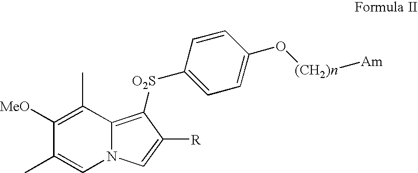

wherein:

n is 3, 4 or 5;

R is optionally substituted alkyl, e.g., methyl, ethyl, isopropyl, butyl, isobutyl; optionally substituted alkenyl; optionally substituted alkynyl; optionally substituted cycloalkyl; optionally substituted heteroalkyl; optionally substituted aryl; optionally substituted heteroaryl; or acyl; or any of the groups listed below for R in Table 1; and

Am is the residue of an amine, such as an optionally substituted alkylamine, and wherein Am is optionally selected from one of the groups listed for Am in Table 2 below, or is optionally selected from the groups listed in Table 3 below.

In another embodiment, a compound of Formula III is provided:

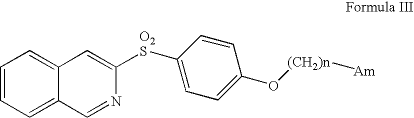

wherein:

n is 3, 4 or 5; and

Am is a residue of an amine, such as an optionally substituted alkyl amine, or Am is optionally selected from the groups listed below in Table 2 or 3.

In one embodiment of a compound of Formula I or II, R is selected from the groups listed below in Table 1.

TABLE 1
R

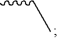

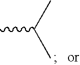

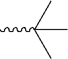

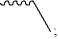

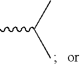

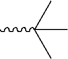

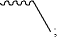

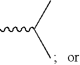

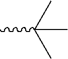

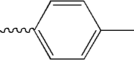

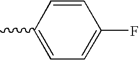

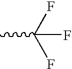

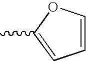

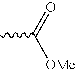

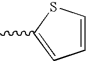

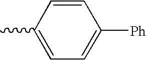

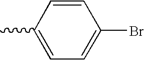

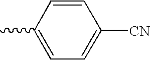

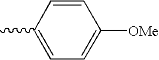

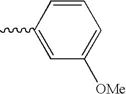

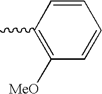

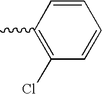

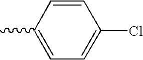

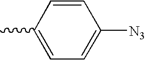

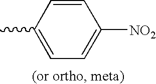

In one embodiment of a compound of Formula I, II, or III, Am is selected from the groups listed below in Table 2 or from the groups listed below in Table 3:

TABLE 2
Am

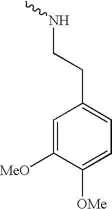

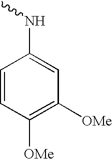

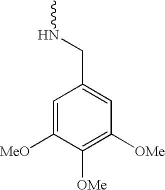

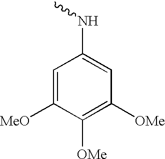

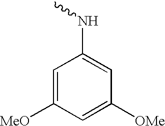

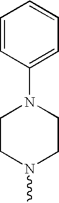

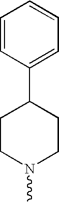

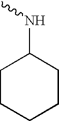

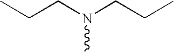

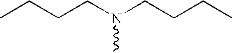

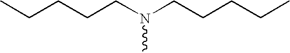

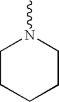

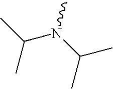

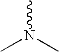

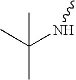

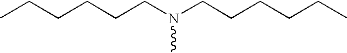

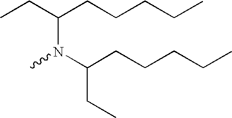

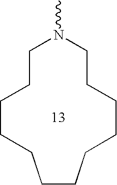

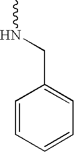

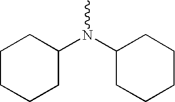

TABLE 3

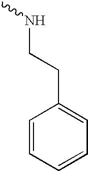

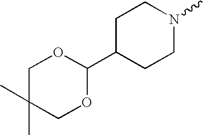

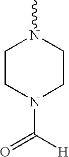

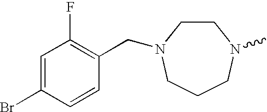

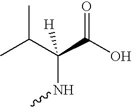

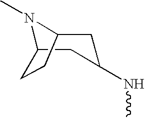

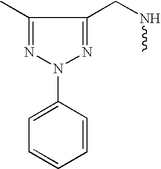

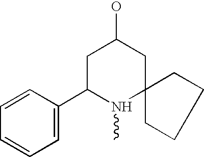

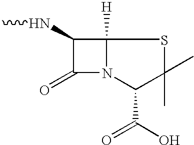

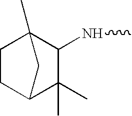

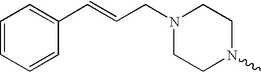

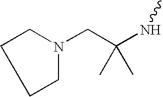

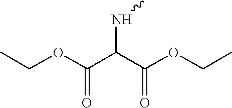

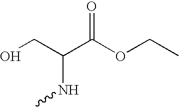

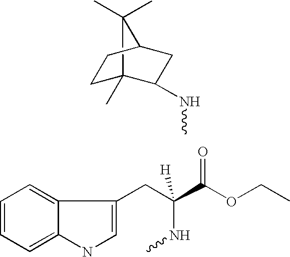

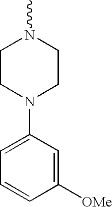

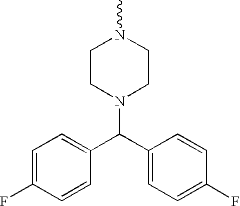

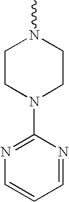

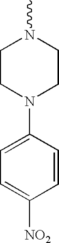

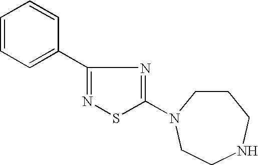

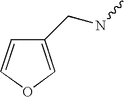

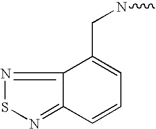

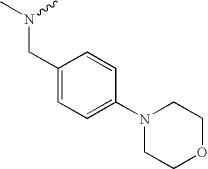

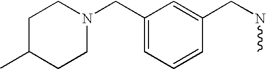

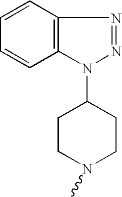

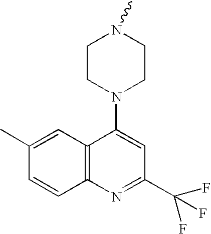

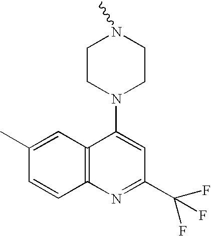

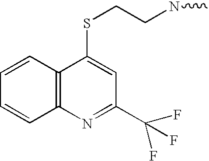

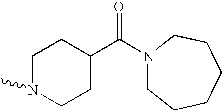

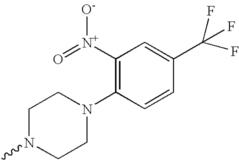

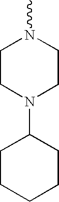

In another embodiment, the compound is a compound of Formula IV, or a salt, ester or prodrug thereof:

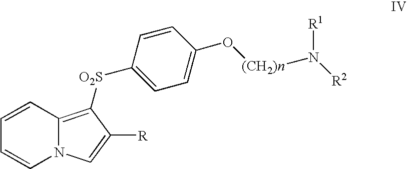

wherein n is, e.g., 1, 2 or 3;

wherein R is optionally substituted alkyl, optionally substituted alkenyl or optionally substituted alkynyl, or in one embodiment, a C1-10 alkyl, e.g. methyl, ethyl, propyl, butyl, secbutyl, isopropyl, or isobutyl;

wherein R¹ is H, unsubstituted alkyl or substituted alkyl, such as C1, C2, C3, C4, C5, C6, C7, C8, C9, C10 unsubstituted or substituted alkyl, including straight chain, branched or cycloalkyl or is the same as R

wherein R² is unsubstituted alkyl or substituted alkyl, such as C1, C2, C3, C4, C5, C6, C7, C8, C9, C10 unsubstituted or substituted alkyl, including straight chain, branched or cycloalkyl, wherein the substituent is, e.g., a substituted or unsubstituted aromatic group; and

wherein optionally R¹ and R² together form an optionally substituted heterocycle, such as an optionally substituted 5, 6, 7, 8, 9, 10, 11, 12, 13 or 14 membered ring saturated or unsaturated heterocycle, including one or more heteroatoms such as nitrogen.

In one subembodiment, in the compound of Formula IV:

n is 1, 2 or 3;

R is methyl, ethyl, propyl, or isopropyl;

R¹ is independently H or C1-C10 unsubstituted alkyl, e.g. methyl, or is the same as

R² is C1-6 unsubstituted or substituted alkyl, wherein, if substituted, the substituent is, e.g., a substituted or unsubstituted aromatic group, such as benzyl optionally substituted with one, two or three halo or alkoxy groups, such as methoxy groups.

In another embodiment, exemplary compounds include the compounds shown below or a salt thereof such as an oxalate salt:

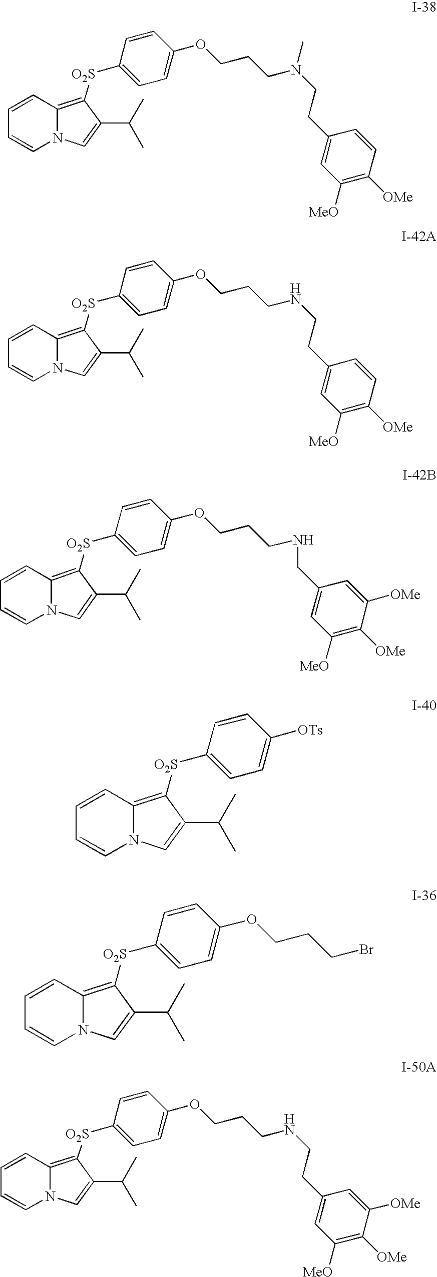

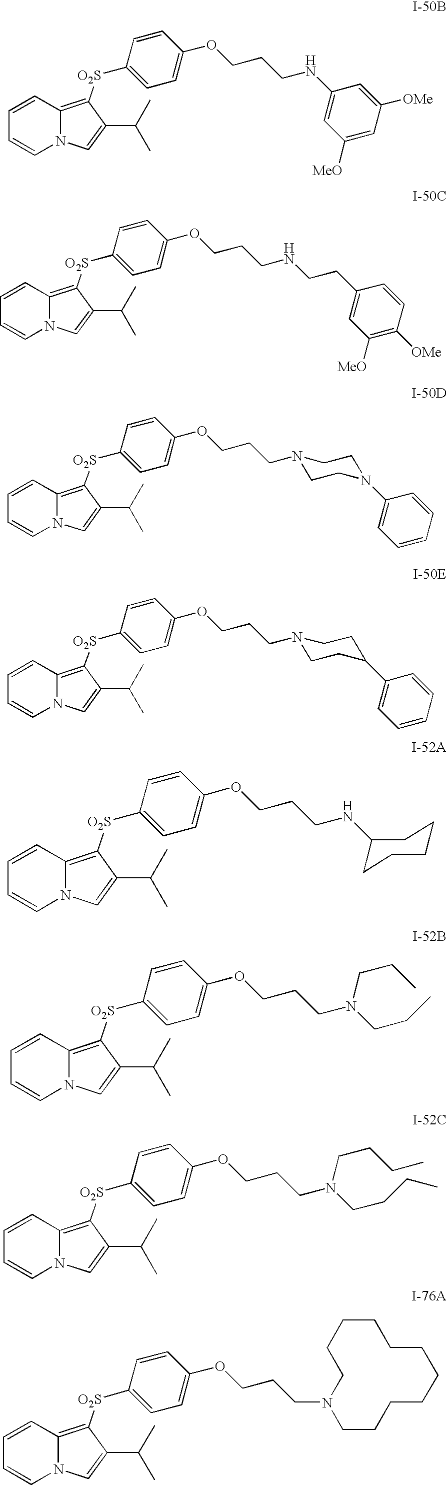

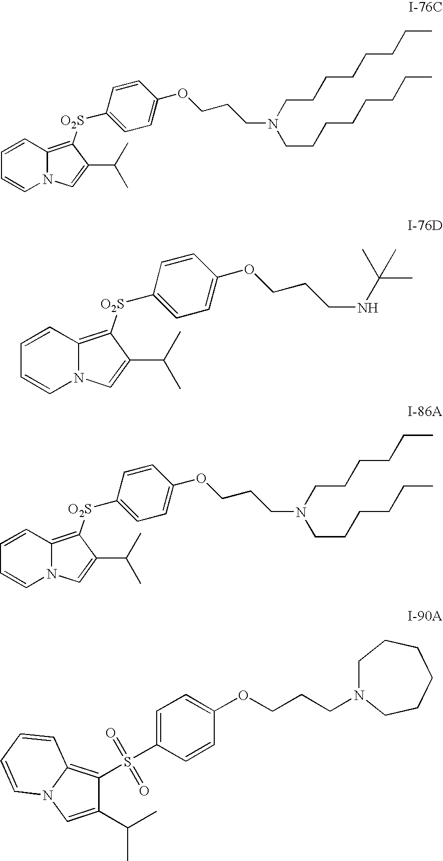

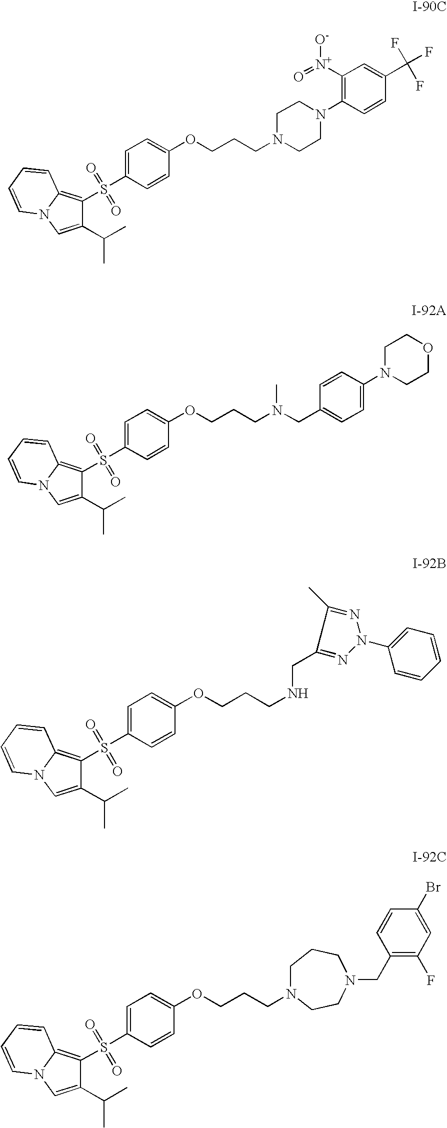

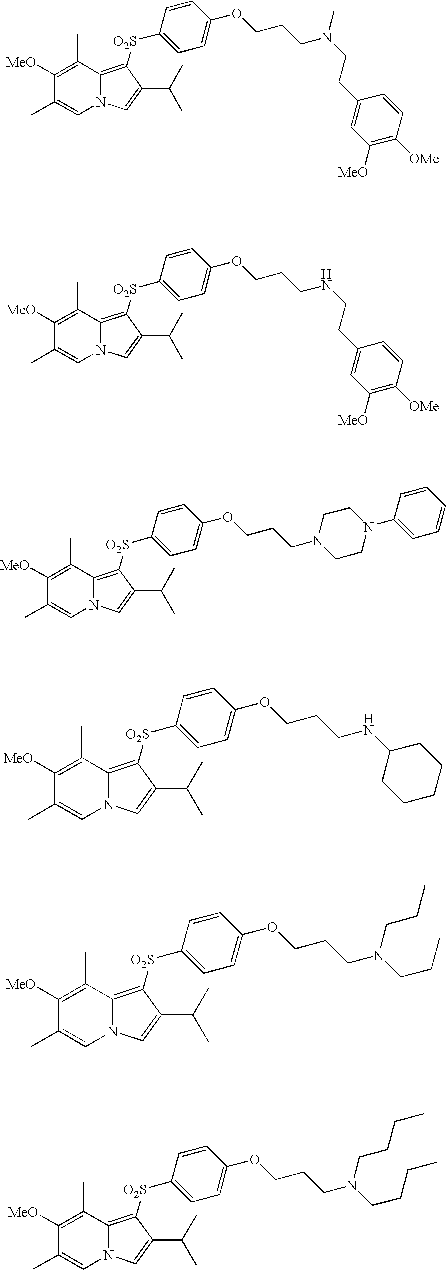

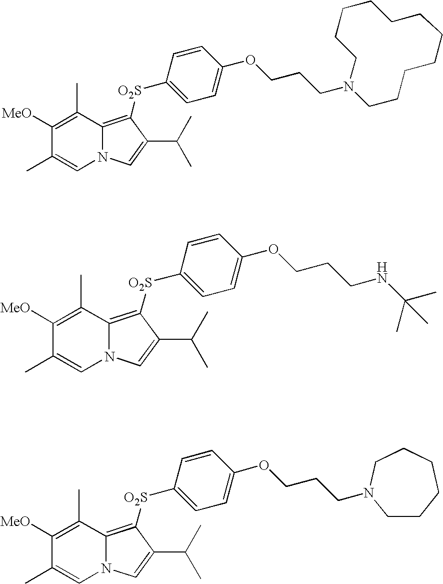

The compounds are optionally in the form of a salt, such as an oxalate salt, or other salt disclosed herein, such as a cesium, hydrochloride, or sulfate salt. The compounds are optionally in the form of a pharmaceutically acceptable salt, ester or prodrug.

Certain compounds disclosed herein can exist in unsolvated forms as well as solvated forms, including hydrated forms. In general, the solvated forms are equivalent to unsolvated forms and are intended to be encompassed within the scope of the present invention. Certain compounds may exist in multiple crystalline or amorphous forms. In general, all physical forms are equivalent for the uses contemplated by the present invention and are intended to be within the scope of the present invention.

Certain compounds disclosed herein possess asymmetric carbon atoms (optical centers) or double bonds; the racemates, diastereomers, geometric isomers and individual isomers are all intended to be encompassed within the scope of the present invention.

The compounds disclosed herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

In one embodiment, optionally, the compound reduces B amyloid production, for example, by at least about 5%, 10%, 15%, 20% or more, in cells that overexpress APP or a fragment thereof.

It is to be understood that the compounds disclosed herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the disclosure of a compound herein encompasses any racemic, optically active, polymorphic, or steroisomeric form, or mixtures thereof, which preferably possesses the useful properties described herein, it being well known in the art how to prepare optically active forms and how to determine activity using the standard tests described herein, or using other similar tests which are will known in the art. Examples of methods that can be used to obtain optical isomers of the compounds include the following:

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme

iv) enzymatic asymmetric synthesis, a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography, a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography, a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent; and

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

Optionally the compound is enantiomerically enriched.

### Methods of Treatment

Methods are provided for treating an animal or human afflicted with a disease associated with cerebral accumulation of Alzheimer's amyloid, such as Alzheimer's disease (AD), comprising administering a therapeutically effective amount of a compound disclosed herein, or a salt, ester or prodrug thereof that is optionally pharmaceutically acceptable.

Administration of the compound in one embodiment results in one or more of reducing β-amyloid production, β-amyloid deposition, β-amyloid neurotoxicity (including abnormal hyperphosphorylation of tau) or microgliosis, or combination thereof. Optionally, the compound is characterized in that it reduces β-amyloid production for example by at least about 5%, 10%, 15%, 20%, 25%, 30%, 50%, or more in cells that overexpress APP or a fragment thereof, as measured, for example, in a culture medium comprising the cells or as measured intracellularly.

As used herein, reference to a compound that reduces β-amyloid production, refers to a compound that reduces β-amyloid production in cells that overexpress APP or a fragment thereof, and the cells may be for example Chinese hamster ovary (CHO) cells that overexpress APP, for example, 7W WT APP751 CHO cells; 7W (wt APP₇₅₁) cells; 7W_{ΔC} cells; 7W_{SW} cells; or 7W_{VF} cells.

It is noted that wherever the embodiments disclosed herein refer to a reduction in β-amyloid in cells that overexpress APP, alternatively, an increase in αCTF (α C-terminal APP fragment, also known as CTF-α) and/or APPSα soluble fragment can be measured for example, in the cell culture or intracellularly, when they are produced in increased amounts from APP as the compound causes the production of β-amyloid to decrease.

It is further noted that wherever the embodiments disclosed herein refer to a reduction in β-amyloid in cells that overexpress APP, alternatively, a decrease in β CTF (β C-terminal APP fragment, also known as CTF-β) or APPSβ soluble fragment can be measured, e.g., in the cell culture media or intracellularly, when they are produced in decreased amounts from APP as the compound causes the production of β-amyloid to decrease.

In a further embodiment, a method is provided for treating animals or humans suffering from traumatic brain injury (TBI). In one embodiment, β-amyloid production, β-amyloiddeposition, β-amyloid neurotoxicity (including abnormal hyperphosphorylation of tau) and/or microgliosis is reduced. The method includes administering to the animal or human, for example, immediately after the TBI, a therapeutically effective amount of a compound disclosed herein, or a salt, ester or prodrug thereof that is optionally pharmaceutically acceptable. The method may include continuing treatment with the compound for a prescribed period of time thereafter. It has been shown that TBI increases the susceptibility to the development of AD, and thus it is believed, without being bound by the theory, that TBI accelerates brain β-amyloid accumulation and oxidative stress, which may work synergistically to promote the onset or drive the progression of AD. Thus, the compound also may decrease β-amyloid production as disclosed herein. Treatment with the compound of animals or humans suffering from a TBI can continue, for example, for about one hour, 24 hours, a week, two weeks, 1-6 months, one year, two years or three years.

Amyloidogenic diseases which can be treated according to the methods of the present invention can include, without limitation, Alzheimer's disease, cerebral anyloid angiopathy, hereditary cerebral hemorrhage with amyloidosis Dutch-type, or other forms of familial AD and familial cerebral Alzheimer's amyloid angiopathy.

The methods of the present invention can be used on transgenic animal models for AD, such as, without limitation, PDAPP and TgAPPsw mouse models, which can be useful for treating, preventing and/or inhibiting conditions associated with β-amyloid production, β-amyloid deposition, β-amyloid neurotoxicity (including abnormal hyperphosphorylation of tau) and microgliosis in the central nervous system of such animals or in humans. Transgenic animal models for AD can be constructed using standard methods known in the art, as set forth for example, without limitation, in U.S. Pat. Nos. 5,487,992; 5,464,764; 5,387,742; 5,360,735; 5,347,075; 5,298,422; 5,288,846; 5,221,778; 5,175,385; 5,175,384; 5,175,383; and 4,736,866.

Exemplary dosages of compound that can be administered include 0.001-1.0 mg/kg body weight. An exemplary dose of compound is about 1 to 50 mg/kg body weight per day, 1 to 20 mg/kg body weight per day, or 0.1 to about 100 mg per kilogram body weight of the recipient per day. Lower doses may be preferable, for example doses of 0.5-100 mg, 0.5-50 mg, 0.5-10 mg, or 0.5-5 mg per kilogram body weight per day, or e.g., 0.01-0.5 mg per kilogram body weight per day. The effective dosage range can be calculated based on the activity of the compound and other factors known in the art of pharmacology.

The compound is conveniently administered in any suitable dosage form, including but not limited to one containing 1 to 3000 mg, or 10 to 1000 mg of active ingredient per unit dosage form. An oral dosage of 50-1000 mg is possible. Lower doses may be preferable, for example from 10-100 or 1-50 mg, or 0.1-50 mg, or 0.1-20 mg or 0.01-10.0 mg. Furthermore, lower doses may be utilized in the case of administration by a non-oral route, as, for example, by injection or inhalation.

In another embodiment, the dosage can range from about 0.05 mg to 20 mg per day, from between about 2 mg to 15 mg per day, about 4 mg to 12 mg per day, and or about 8 mg per day.

In another embodiment, the dosage ranges, e.g. from about one day to twelve months, from about one week to six months, or from about two weeks to four weeks.

Because most diseases having cerebral accumulation of Alzheimer's amyloid, such as AD, are chronic, progressive, intractable brain dementias, it is contemplated that the duration of treatment with compounds disclosed herein can last for up to the lifetime of the animal or human.

### In Vitro Assay Methods

In vitro assay methods available in the art may be used to assay the compounds for activity in inhibiting β-amyloid and for usefulness in the treatment of diseases associated with β-amyloid overproduction and/or accumulation. In one embodiment, an assay to determine the compounds' ability to decrease β-amyloid production is conducted. For example, the test compound is exposed to cells that overexpress APP or a fragment thereof; β-amyloid production in the cells is measured; and a decrease in β-amyloid production of e.g., at least about 20% more in the cells that overexpress APP or a fragment thereof is detected as an indicator of the therapeutic usefulness of the compound to treat animals or humans afflicted with a disease associated with cerebral accumulation of Alzheimer's amyloid. The assay is conducted using cells that overexpress APP or a fragment thereof available in the art such as Chinese hamster ovary cells that overexpress APP751. The β-amyloid measured, is, e.g., Aβ1-40, Aβ1-42, or total Aβ1-40+Aβ1-42. A decrease in the production of Aβ1-40 and/or Aβ1-42, and in particular, total Aβ1-40+Aβ1-42, of, e.g., at least about 5%, 10%, 15%, 20%, 25%, 30%, 50%, or more, indicates the therapeutic effectiveness of the compound to treat animals or humans afflicted with a disease associated with cerebral accumulation of Alzheimer's amyloid. The S-amyloid concentrations can be measured for example, intracellularly or, e.g., extracellularly in the culture medium.

The compounds can be screened in a range of concentrations, for example, about 1 nM to 10 mM, about 500 nM to 50 μM, or about 5 μM to 30 μM.

In one embodiment, the cells to be tested for a reduction in β-amyloid production in cells are exposed to the test compound. In the method, the concentration of β-amyloid (e.g., Aβ1-40 and/or Aβ1-42) in cells exposed to the compound can be measured and compared with a measurement of β-amyloid production in unexposed cells, for example, in a control run in parallel. A decrease in the production β-amyloid, alone or in combination, for example of about 5%, 10%, 15%, 20%, 25%, 30%, 50%, or more in the exposed cells compared to the control cells indicates the potential therapeutic effectiveness of the compound to treat animals or humans afflicted with a disease associated with cerebral accumulation of Alzheimer's amyloid. Optionally, total β-amyloid concentration (Aβ1-40+Aβ1-42) is measured. The β-amyloid is measured, e.g. in the culture medium comprising the cells, or intracellularly.

The method of measuring β-amyloid may include testing an array of compounds, e.g., in a 24 well plate or a 96 well plate, as well as one or more control samples. In the assay, the compound is often required to be incubated with the cells for about 4-48 hours, or e.g., 18-36 hours. β-amyloid can be detected using an ELISA sandwich assay using quantitatively commercially available enzymatically labeled (with horseradish peroxidase) antibodies to Aβ1-40 and Aβ1-42 as described in the Examples. The labeled antibody ELISA assay also can require on the order of 24 hours to complete.

Cells which can be used in assays for measuring a reduction in β-amyloid production include mammalian or non-mammalian cells that overexpress APP or a fragment thereof, including but not limited to Chinese hamster ovary (CHO) cells, for example, 7W WT APP751 CHO cells. See, e.g., Koo and Squazzo, J. Biol. Chem., Vol. 269, Issue 26, 17386-17389, July, 1994. Cell lines transfected with APP have been described in the art and include 7W (wt APP₇₅₁); 7W_{ΔC} (APP₇₅₁ with deletion of almost the entire cytoplasmic tail (residue 710-751); 7W_{SW} (APP₇₅₁ with the “Swedish” KM651/652NL double-mutation); and 7W_{VF} (APP₇₅₁ with the V698F mutation). See, e.g. Xia et al., Proc. Natl. Acad. Sci. USA, Vol. 94, pp. 8208-8213, July 1997; and Perez, R. & Koo, E. (1997) in Processing of the β-Amyloid Precursor Protein: Effects of C-Terminal Mutations on Amyloid Production, eds. Iqbal, K., Winblad, B., Nishimura, T., Takeda, M. & Wisniewski, H. M. (J. Wiley & Sons, London), pp. 407-416. The APP which is overexpressed can include transcripts of APP, such as, without limitation, APP751.

### Methods of Diagnosis

In still a further embodiment, a method is provided for diagnosing or determining the risk for developing a disease associated with cerebral accumulation of Alzheimer's amyloid, such as AD, in an animal or human, by taking a first measurement of β-amyloid concentration from a peripheral body fluid such as plasma, serum, whole blood, urine or cerebral spinal fluid (CSF) of the animal or human. Subsequently the method includes administering to the animal or human a diagnostically effective amount of a compound as disclosed herein. Optionally, the compound is one that decreases β amyloid production for example by at least about 5%, 10%, 15%, 20%, 25%, 30%, 50%, or more, as measured, for example, in the medium of cultured cells which overexpress APP or a fragment thereof, or as measured intracellularly. A second (selected endpoint) measurement of β-amyloid concentration is taken from plasma, serum, whole blood, urine or CSF of the animal or human at a later time, and the difference between the first measurement and the second measurement is determined. A change in the concentration of β-amyloid in plasma, serum, whole blood, urine or CSF in the second measurement compared to the first measurement indicates a risk of developing or a possible diagnosis of a disease associated with cerebral accumulation of Alzheimer's amyloid in the animal or human. In one embodiment, an increase or decrease in peripheral β-amyloid indicates the presence of an accumulation of cerebral β-amyloid, and therefore the risk of disease or the presence of the disease.

It is believed, without being bound by any theory, that the compounds can cause a change in β-amyloid concentration in plasma, urine, serum, whole blood or CSF.

The duration of time of administration of the compound after the first peripheral body fluid measurement, up until the second (selected endpoint) peripheral body fluid measurement, is, e.g., any suitable time period, e.g. about 1-12 hours, about 1-7 days, about 1-4 weeks; about 2-6 months, or more. The time length can be adjusted as needed depending, for example, on the progression of the disease, and the patient. A suitable periodic (e.g., daily) dosage of the compound is administered, e.g. orally or intravenously, and the β-amyloid levels in the individual can be monitored periodically up until the endpoint. In one preferred embodiment, the compound is administered daily for about 3 days to 4 weeks from the start of administration to the endpoint measurement. The change in concentration indicative of the risk or presence of a disease associated with β-amyloid accumulation is, e.g. about 10-20% or more between the first and endpoint measurements.

Exemplary dosages of compound that can be administered include 0.001-1.0 mg/kg body weight, for example daily. An exemplary dose of compound is about 1 to 50 mg/kg body weight per day, 1 to 20 mg/kg body weight per day, or 0.1 to about 100 mg per kilogram body weight of the recipient per day. Lower doses may be preferable, for example doses of 0.5-100 mg, 0.5-50 mg, 0.5-10 mg, or 0.5-5 mg per kilogram body weight per day, or e.g., 0.01-0.5 mg per kilogram body weight per day. The effective dosage range can be calculated based on the activity of the compound and other factors known in the art of pharmacology.

The compound is conveniently administered in any suitable dosage form, including but not limited to one containing 1 to 3000 mg, or 10 to 1000 mg of active ingredient per unit dosage form. An oral dosage of 50-1000 mg is possible. Lower doses may be preferable, for example from 10-100 or 1-50 mg, or 0.1-50 mg, or 0.1-20 mg or 0.01-10.0 mg. Furthermore, lower doses may be utilized in the case of administration by a non-oral route, as, for example, by injection or inhalation.

### Synthesis of Compounds

The compounds disclosed herein can be made using synthetic techniques available in the art.

A general synthetic scheme is shown below:

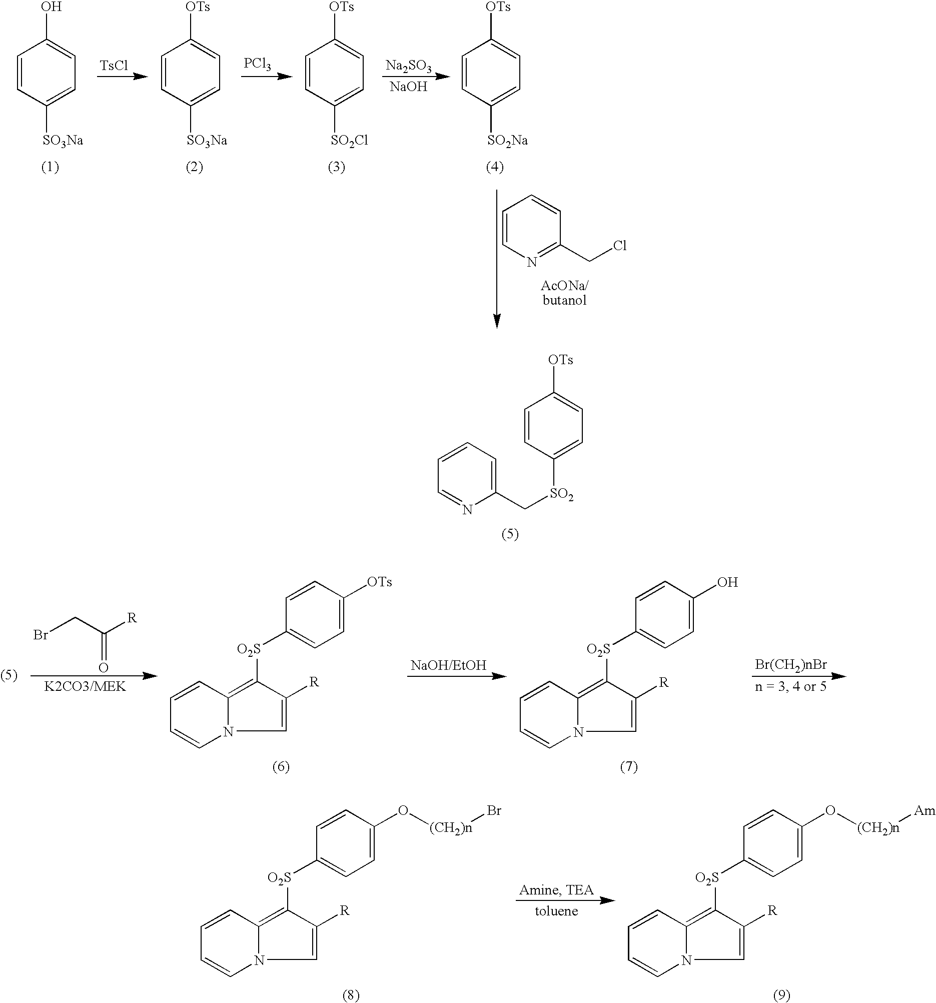

Sodium p-Tosylated benzenesulfate. To the sodium benzenesulfate dissolved in 1M NaOH is added tosyl chloride (2 eq.) and the reaction heated on a steam bath for 2.5 hours. The reaction then is acidified with 3N hydrochloric acid and then added to ice, filtered and washed well with water, then cold ether to afford the desired product as a white solid.

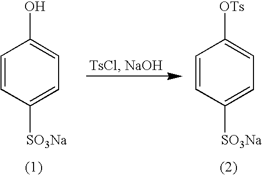

p-Tosylated benzenesulfonyl chloride. To powered sodium p-tosylated benzenesulfate is added phosphorus pentachloride (e.g., 2 eq.) in small portions. The resulting product is heated in an oil bath at 110° C. for 10 hours. The mixture is then washed with water and allowed to dry.

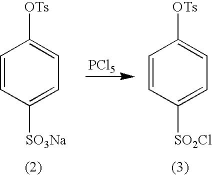

Sodium p-Tosylated benzenesulfinate. To a solution of sodium sulfite (3 eq) in 1M sodium hydroxide is added p-tosylated benzenesulfonyl chloride and the reaction allowed to stir e.g., for 3 h. The reaction is then filtered and the compound air-dried, for example, overnight.

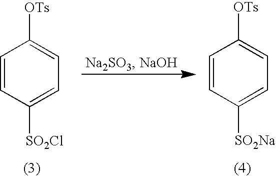

4-(Tosyloxy)phenyl 2-picolyl sulfone. To a solution of sodium p-tosylated benzenesulfinate in n-butanol is added picolyl chloride (1.5 eq) and sodium acetate (2 eq) and the reaction refluxed overnight. The reaction is then allowed to cool and poured onto water and the mixture stirred for, e.g., fifteen minutes upon which it is filtered and washed with water and allowed to air dry.

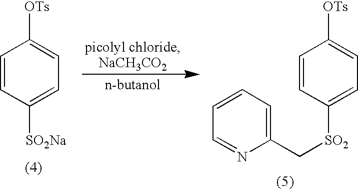

2-Isopropyl-1-[[4-(tosyloxy)phenyl]sulfonyl]indolizine. To a solution of 4-Tosyloxy)phenyl 2-picolyl sulfone in an organic solvent is added 1-bromo-3-methyl-2-butanone (2 eq) and K₂CO₃ (2 eq). The mixture is refluxed e.g. for 24 h. The reaction medium is then brought back to room temperature, evaporated under vacuum to remove the excess ketone and extracted with e.g. ethyl acetate:brine (1:1). The organic layer is dried over sodium sulfate and evaporated under vacuum leaving a solid which is purified, e.g., by column chromatography.

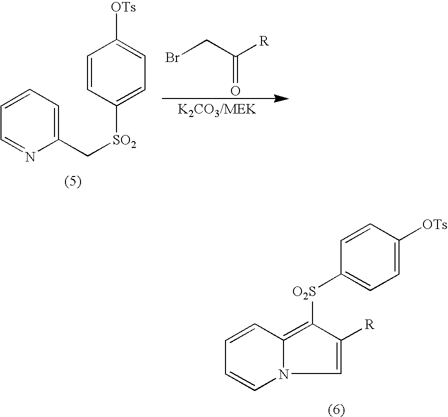

2-Isopropyl-1-([4-hydroxyphenyl)sulfonyl]indolizine. 2-Isopropyl-1-[[4-(tosyloxy)phenyl]sulfonyl]indolizine is added to an ethanol/water mixture containing NaOH (2M) and the mixture refluxed for 24 h. After cooling, the solution is further diluted with more water and then extracted with ether. The aqueous layer is acidified and extracted with ethyl acetate, dried over sodium sulfate and concentrated to dryness under vacuum.

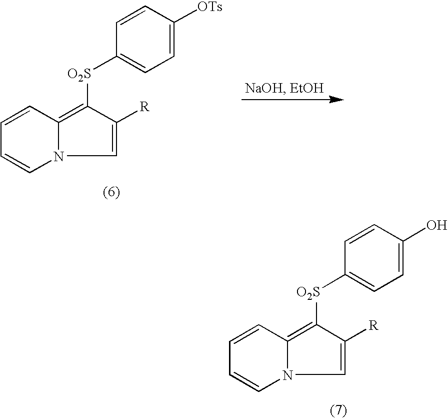

2-Isopropyl-1-[[4-[(3-bromopropyl)oxy]-phenyl]sulfonyl]indolizine. To a solution of 2-isopropyl-1-([4-hydroxyphenyl)sulfonyl]indolizine in methyl ethyl ketone is added K₂CO₃ (1.5 eq) and 1,3-dibromopropane (5 eq); the mixture is brought to reflux e.g. for 24 h. After the reaction, the solution is evaporated to dryness and extracted. The organic layer is dried over sodium sulfate and concentrated under vacuum and the residue is purified by e.g. chromatography. The homogeneous fractions are pooled and evaporated to dryness.

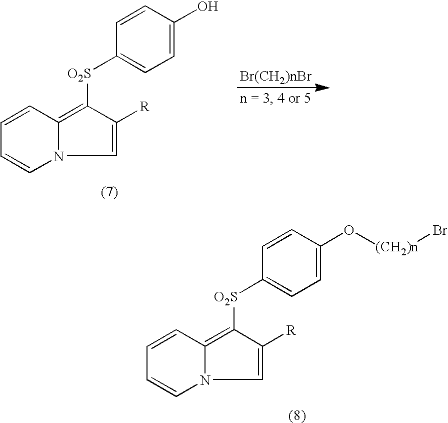

Compounds. 2-Isopropyl-1-[[4-[(3-bromopropyl)oxy]-phenyl]sulfonyl]indolizine, N-methyl-N-(3,4-dimethoxyphenethyl)amine (2.5 eq) or derivative thereof, and triethylamine (1.5 eq) are refluxed in toluene for 24 h. The solvent is then eliminated under vacuum and the residue is taken up in H₂O. The medium is extracted with a solvent such as CH₂Cl₂, dried over Na₂SO₄ and evaporated to dryness. The oil obtained is purified via flash chromatography.

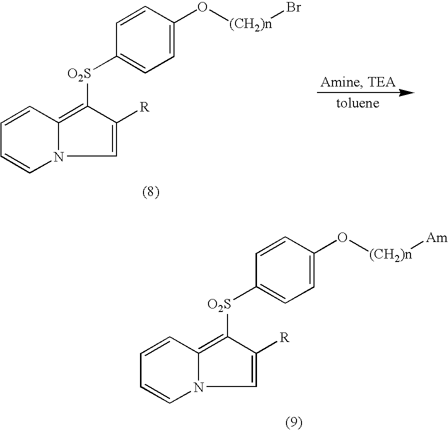

Oxalate Salts of compounds. The compounds are optionally isolated as a salt such as an oxalate salt. To a solution of the base dissolved in acetone is added a stoichiometric amount of oxalic acid and the corresponding oxalates are recrystallized from acetone or isopropanol/hexane mixture.

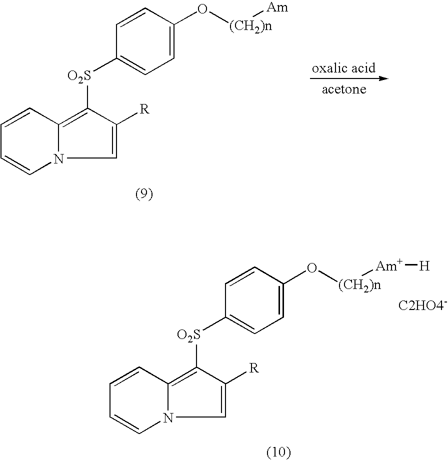

Using the above procedures, e.g., with the appropriate reagent to substitute for the amine (Am), the compounds disclosed herein can be prepared.

In another embodiment, the transformation of compound (3) to compound (5) can be implemented in a one step reaction as shown below, which can be conducted in an aqueous solvent:

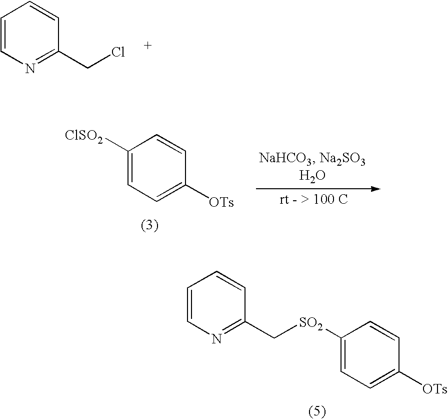

In another embodiment, the reagents are modified and the reaction can be conducted as shown below:

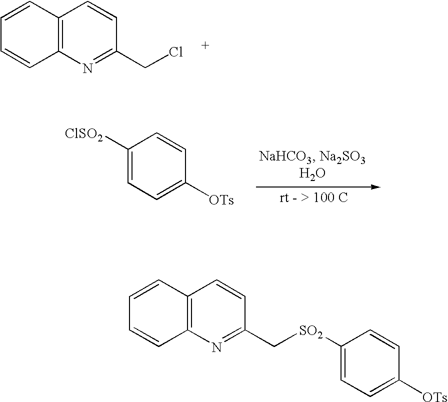

Thus, in one embodiment, a method is provided which comprises reacting a heterocycle comprising a 2-chloromethyl group (such as 2-(chloromethyl)quinoline) with a sulfonyl chloride, such as a benzene sulfonyl chloride (such as p-tosylated benzenesulfonyl chloride) to form a sulfone linkage between the methyl group on the heterocycle and the sulfonyl chloride. The reaction is conducted in aqueous solvent in the presence of sodium bicarbonate and sodium sulphite.

In one embodiment, the reaction is represented by:

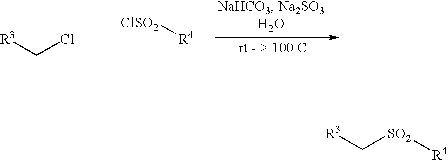

wherein R³ is e.g., a heterocycle that is optionally substituted, and R⁴ is, e.g., optionally substituted alkyl, alkenyl, alkynyl, aryl, heterocycle, or heteroaryl.

### Pharmaceutical Formulations and Methods of Administration

Compounds disclosed herein can be administered in an effective amount for the treatment of a disease associated with cerebral accumulation of β-amyloid, such as Alzheimer's disease, cerebral amyloid angiopathy, hereditary cerebral hemorrhage with amyloidosis Dutch-type, other forms of familial Alzheimer's disease and familial cerebral Alzheimer's amyloid angiopathy. Such compounds are also referred to herein as “active agents”. Dosage amounts and pharmaceutical formulations can be selected using methods known in the art. The compound can be administered by any route known in the art including parenteral, oral or intraperitoneal administration.

The compounds disclosed herein that are administered to animals or humans are dosed in accordance with standard medical practice and general knowledge of those skilled in the art. In particular, therapeutically effective amounts of compounds or more, can be administered in unit dosage form to animals or humans afflicted with a disease associated with cerebral accumulation of Alzheimer's amyloid or suffering from a traumatic brain injury, as well as administered diagnostically for the purpose of determining the risk of developing and/or a diagnosis of a disease associated with cerebral accumulation of Alzheimer's amyloid.

Parenteral administration includes the following routes: intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, or nasal inhalation via insufflation or nebulization. The nasal inhalation is conducted, for example, using aerosols, atomizers or nebulizers.

Examples of suitable dosage amounts are, e.g., about 0.02 mg to 1000 mg per unit dose, about 0.5 mg to 500 mg per unit dose, or about 20 mg to 100 mg per unit dose. The daily dosage can be administered in a single unit dose or divided into two, three or four unit doses per day. The duration of treatment of the active agent is, for example, on the order of hours, weeks, months, years or a lifetime. The treatment may have a duration, for example, of 1-7 days, 1-4 weeks, 1-6 months, 6-12 months, or more.

The compound can be administered to the CNS, parenterally or intraperitoneally. Solutions of compound e.g. as a free base or a pharmaceutically acceptable salt can be prepared in water mixed with a suitable surfactant, such as hydroxypropylcellulose. Dispersions also can be prepared in glycerol, liquid polyethylene glycols, and mixtures thereof, and in oils. Under ordinary conditions of storage and use, these preparations can contain a preservative and/or antioxidants to prevent the growth of microorganisms or chemical degeneration.

The compounds which are orally administered can be enclosed in hard or soft shell gelatin capsules, or compressed into tablets. The compounds also can be incorporated with an excipient and used in the form of ingestible tablets, buccal tablets, troches, capsules, sachets, lozenges, elixirs, suspensions, syrups, wafers, and the like. Further, compounds can be in the form of a powder or granule, a solution or suspension in an aqueous liquid or non-aqueous liquid, or in an oil-in-water or water-in-oil emulsion.

The tablets, troches, pills, capsules and the like also can contain, for example, a binder, such as gum tragacanth, acacia, corn starch; gelating excipients, such as dicalcium phosphate; a disintegrating agent, such as corn starch, potato starch, alginic acid and the like; a lubricant, such as magnesium stearate; a sweetening agent, such as sucrose, lactose or saccharin; or a flavoring agent. When the dosage unit form is a capsule, it can contain, in addition to the materials described above, a liquid carrier. Various other materials can be present as coatings or to otherwise modify the physical form of the dosage unit. For example, tablets, pills, or capsules can be coated with shellac, sugar or both. A syrup or elixir can contain a compound as disclosed herein, sucrose as a sweetening agent, methyl and propylparabens as preservatives, a dye and flavoring. Additionally, a compound can be incorporated into sustained-release preparations and formulations.

The invention will be understood in further detail in view of the following non-limiting examples.

### EXAMPLES

### Example 1

### Assay for Measurement of Aβ1-40 and/or Aβ1-42

Chinese hamster ovary (CHO) cells, stably transfected with human APP751 (7W WT APP751 CHO cells) are used. See, e.g., Koo and Squazzo, J. Biol. Chem., Vol. 269, Issue 26, 17386-17389, July, 1994. The cells are maintained in DMEM medium supplemented with 10% fetal bovine serum and 1× mixture of penicillin/streptomycin/fungizone/glutamine mixture (Cambrex, Md.) geneticin as selecting agent in 75 cm cell culture flasks.

The 7W WT APP751 CHO cells overexpressing APP751 are plated into 24-well culture plates in 1 mL of culture medium. Each compound is added to confluent cells to a final concentration of e.g. of 30 μM, 10 μM or 3 μM. After 24 hours of treatment, culture medium is collected and dissolved 10-fold and 2-fold for measuring the level of Aβ1-40 and/or Aβ1-42, respectively. The control is 1% DMSO. Aβ1-40 and Aβ1-42 are determined using commercially available ELISAs (Biosource, Calif.), following the recommendations of the manufacturer.

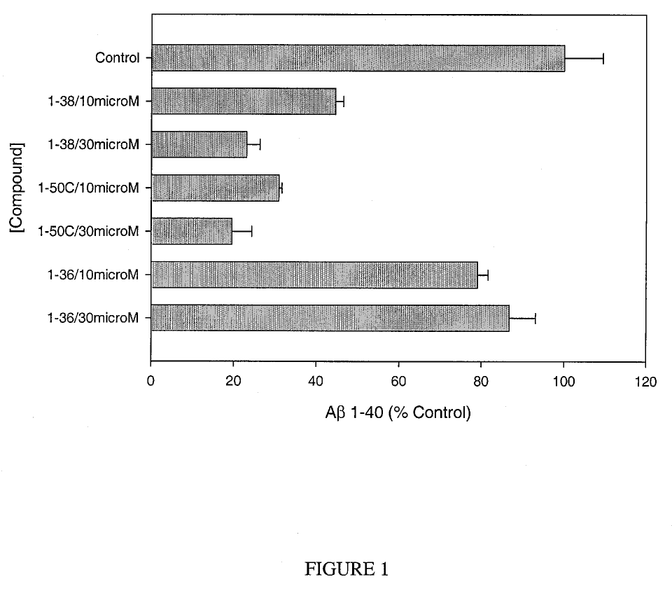

The results of the effect on Aβ1-40 with compounds 1-38, 1-50C and 1-36 at 10 μM and 30 μM are shown in FIG. 1.

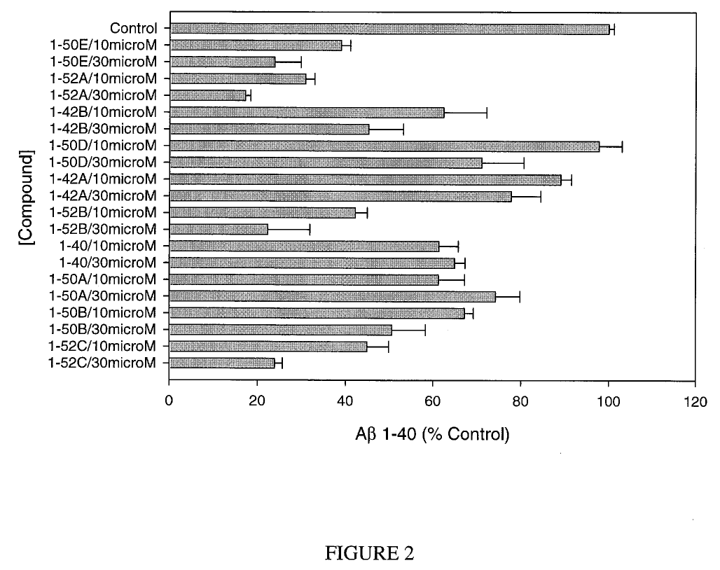

The results of the effect on Aβ1-40 with the compounds: 1-50E; 1-52A; 1-42B; 1-50D; 1-42A; 1-52B; 1-40; 1-50A; 1-50B; and 1-52C at 10 μM and 30 μM are shown in FIG. 2.

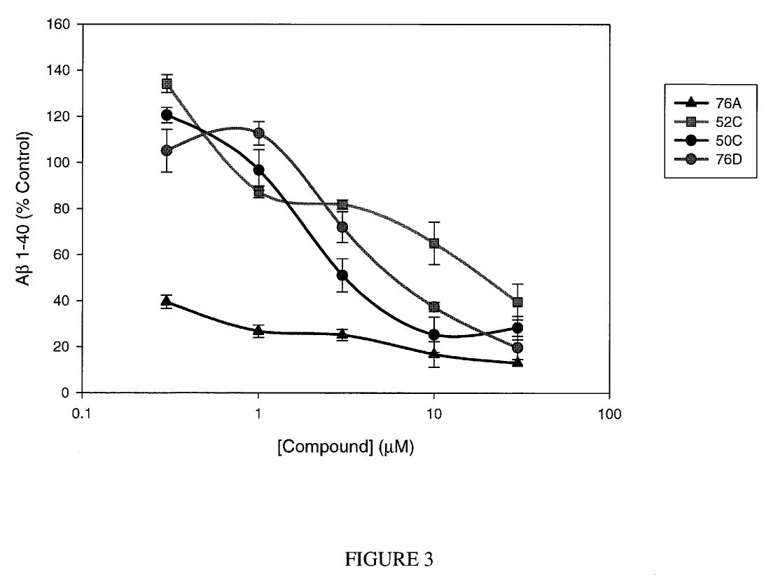

The results of the effect on Aβ1-40 with the compounds: 1-76A; 1-52C; 1-50C and 1-76D at 0.3, 1.0, 3.0, 10.0 and 30 μM are shown in FIG. 3.

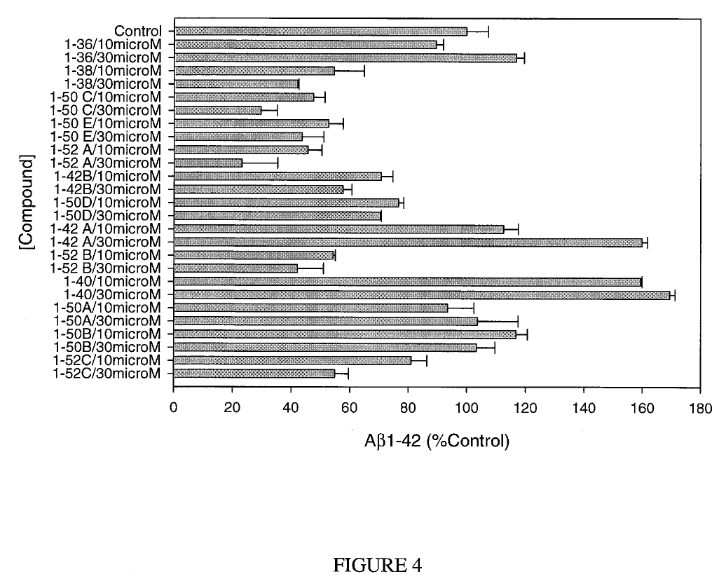

The results of the effect on Aβ1-42 with the compounds: 1-36; 1-38; 1-50C; 1-50E; 1-52A; 1-42B; 1-52B; 1-40; 1-50A; 1-50B; and 1-52C at 10M and 30 μM are shown in FIG. 4.

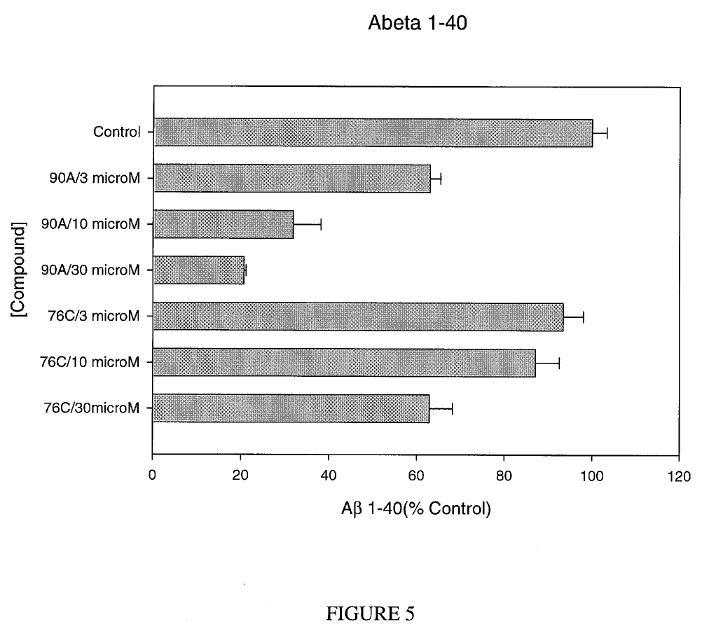

The results of the effect on Aβ1-40 with the compounds: 1-90A; and 1-76C at 3 μM, 10 μM and 30 μM are shown in FIG. 5.

### Example 2

### Synthesis

General techniques: All reactions requiring anhydrous conditions are conducted in oven-dried glass apparatus under an atmosphere of nitrogen. Preparative chromatographic separations are performed on Combiflash Companion, Isco Inc.; reactions are followed by TLC analysis using silica plates with fluorescent indicator (254 nm) and visualized with UV, phosphomolybdic acid or 4-hydroxy-3-methoxybenzaldehyde. All commercially available reagents are purchased from Aldrich and Acros and are typically used as supplied.

Melting points are recorded using open capillary tubes on a Bamstead melting point apparatus and are uncorrected. ¹H and ¹³C NMR spectra are recorded in Fourier transform mode at the field strength specified on a Varian AS500 spectrometer. Spectra are obtained on CDCl₃ solutions in 5 mm diameter tubes, and the chemical shift in ppm is quoted relative to the residual signals of chloroform (δ_{H} 7.25 ppm, or δ_{C} 77.0 ppm). Multiplicities in the ¹H NMR spectra are described as: s=singlet, d=doublet, t=triplet, q=quartet, m=multiplet, br=broad; coupling constants are reported in Hz. Low (MS) resolution mass spectra are measured on a Micromass Q-T of API-US spectrometer utilizing an Advion Bioscience Nanomate electrospray source. Ion mass/charge (m/z) ratios are reported as values in atomic mass units.

A general synthetic scheme is shown below:

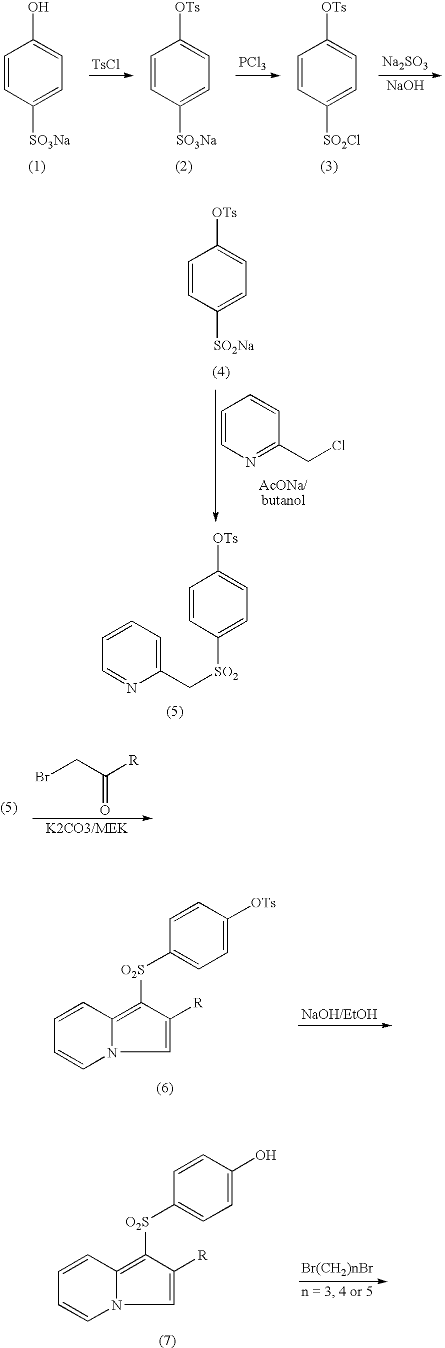

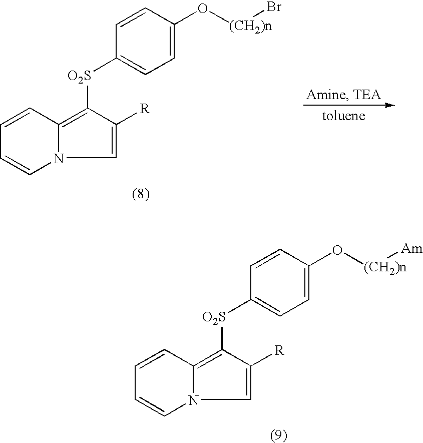

Sodium p-Tosylated benzenesulfate. To the sodium benzenesulfate dissolved in 1M NaOH is added tosyl chloride (2 eq.) and the reaction heated on a steam bath for 2.5 hours. The reaction is then acidified with 3N hydrochloric acid and then added to ice, filtered and washed well with water, then cold ether to afford the desired product as a white solid.

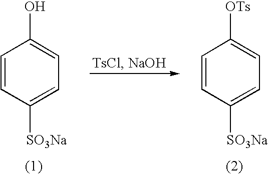

p-Tosylated benzenesulfonyl chloride. To powered sodium p-tosylated benzenesulfate was added finely divided phosphorus pentachloride (2 eq.) in small portions. The first portion reacted at once and the mixture of solids began to liquefy to ensure stirring during the rest of the reaction. When addition was complete, the resulting thin syrup in heated in an oil bath at 110° C. for 10 hours, leaving an almost clear and colorless mixture. The mixture is then added to crushed ice and the lumps were broken up, filtered off, washed well with water and allowed to dry.

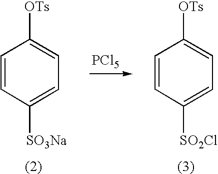

Sodium p-Tosylated benzenesulfinate. To a solution of sodium sulfite (3 eq) in 1M sodium hydroxide is added p-tosylated benzenesulfonyl chloride and the reaction allowed to stir for 3 h. The reaction is then filtered and the compound was air-dried overnight.

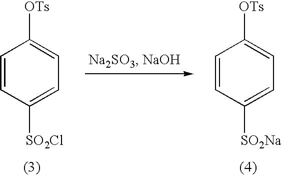

4-(Tosyloxy)phenyl 2-picolyl sulfone. To a solution of sodium p-tosylated benzenesulfinate in n-butanol was added picolyl chloride (1.5 eq) and sodium acetate (2 eq) and the reaction refluxed overnight. The reaction is then allowed to cool and poured onto water and the mixture stirred for fifteen minutes upon which it was filtered and washed with water and allowed to air dry.

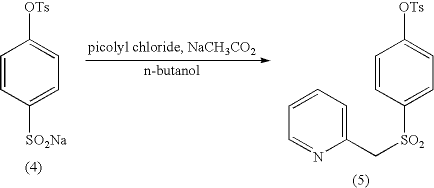

2-Isopropyl-1-[[4-(tosyloxy)phenyl]sulfonyl]indolizine. To a solution of 4-Tosyloxy)phenyl 2-picolyl sulfone in methylethyl ketone or THF is added 1-bromo-3-methyl-2-butanone (2 eq) and K₂CO₃ (2 eq). The mixture is refluxed for 24 h. The reaction medium is then brought back to room temperature, carefully evaporated under vacuum to remove the excess ketone and extracted with ethyl acetate:brine (1:1). The organic layer is dried over sodium sulfate and evaporated under vacuum leaving a yellow solid which is purified by column chromatography using a stepwise elution of 3:7 ethyl acetate:hexane then 1:1 ethyl acetate:hexane.

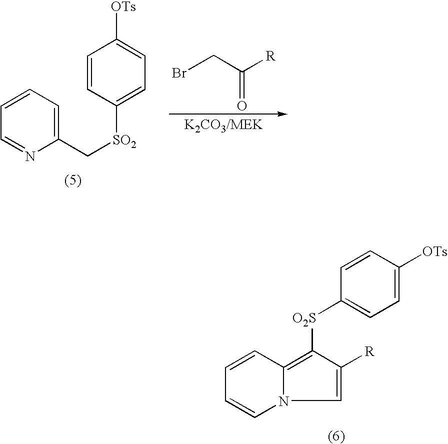

2-Isopropyl-1-([4-hydroxyphenyl)sulfonyl]indolizine. 2-Isopropyl-1-[[4-(tosyloxy)phenyl]sulfonyl]indolizine is added to an ethanol/water mixture containing NaOH (2M) and the mixture was refluxed for 24 h. After cooling, the solution is further diluted with more water and then extracted with ether. The aqueous layer was acidified and extracted with ethyl acetate, dried over sodium sulfate and concentrated to dryness under vacuum.

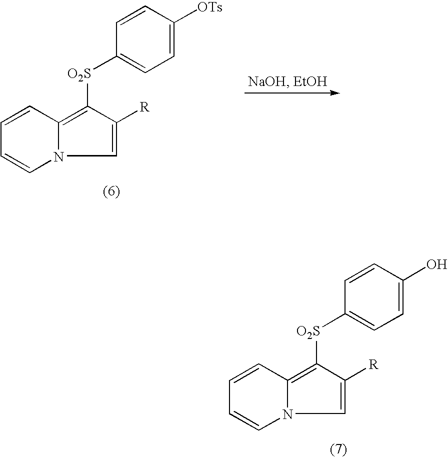

2-Isopropyl-1-[[4-[(3-bromopropyl)oxy]-phenyl]sulfonyl]indolizine. To a solution of 2-isopropyl-1-([4-hydroxyphenyl)sulfonyl]indolizine in methyl ethyl ketone is added K₂CO₃ (1.5 eq) and 1,3-dibromopropane (5 eq); the mixture is brought to reflux for 24 h. After the reaction, the solution is evaporated to dryness and extracted with brine/methylene chloride. The organic layer is dried over sodium sulfate and concentrated under vacuum and the residue is purified by chromatography on a silica column with methylene chloride as the eluent. The homogeneous fractions are pooled and evaporated to dryness.

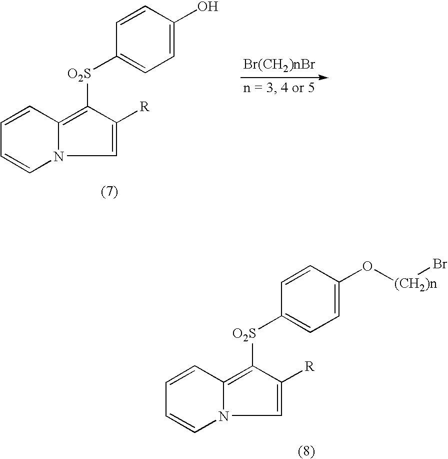

Compounds. 2-Isopropyl-1-[[4-[(3-bromopropyl)oxy]-phenyl]sulfonyl]indolizine, N-methyl-N-(3,4-dimethoxyphenethyl)amine (2.5 eq) or derivative thereof, and triethylamine (1.5 eq) are refluxed in toluene for 24 h. The solvent then is eliminated under vacuum and the residue is taken up in H₂O. The medium is extracted with CH₂Cl₂, dried over Na₂SO₄ and evaporated to dryness. The oil obtained is purified via flash chromatography using CH₂Cl₂/MeOH as the eluent.

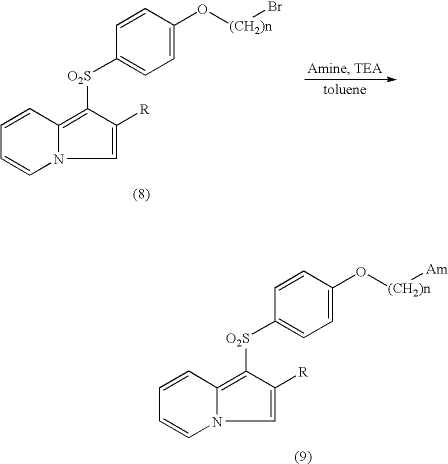

Oxalate Salts. The compounds are isolated as the oxalate salt. To a solution of the base dissolved in acetone is added a stoichiometric amount of oxalic acid and the corresponding oxalates are recrystallized from acetone or isopropanol/hexane mixture.

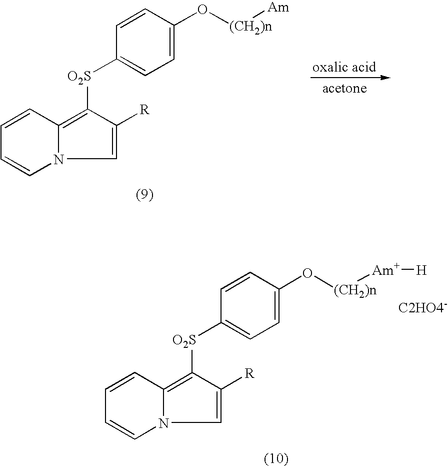

Alternatively, the transformation of compound (3) to compound (5) is implemented in a one step reaction as shown below, which can be conducted in an aqueous solvent:

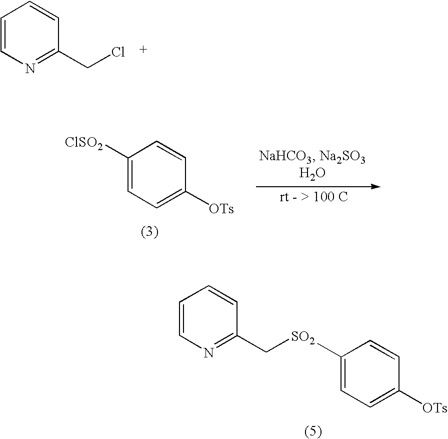

To a solution of NaHCO₃ (4.9 g, 58.38 mmol, 5 equiv.) and Na₂SO₃ (2.94 g, 23.35 mmol, 2 equiv.) in H₂O (20 mL) is slowly added the p-tosylated benzenesulfonyl chloride (by portion and the reaction is stirred at rt (room temperature) for 2 hours. Then the 2-(chloromethyl) containing hetrocyclic compound, e.g. 2-(chloromethyl)quinoline (4.04 g, 11.7 μmmol, 1 equiv.) is added very slowly by portion and the reaction is stirred 10 minutes at rt and then warmed up to 100 C for 5 hours. The reaction is cooled to rt and extracted with DCM several times. The crude product with 99+% is used for the next step without further purification.

Using the above procedures with the appropriate reagent to substitute for the amine (Am) the following compounds were obtained as oxalate salts and chemically identified as noted below:

### {3-[4-(2-Isopropyl-indolizine-1-sulfonyl)-phenoxy]-propyl}-(3,4,5-trimethoxy-phenyl)-amine (1-50A)

2-Isopropyl-1-[[4-[(3-bromopropyl)oxy]-phenyl]sulfonyl]indolizine (8), 3,4,5-Trimethoxyaniline (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

¹H NMR (500 MHz, acetone-d₆) δ 1.24 (d, J=6.9 Hz, 6H), 2.82-2.87 (m, 8H), 3.31 (t, J=6.5 Hz, 2H), 4.20 (t, J=6.1 Hz, 2H), 5.95-6.01 (m, 2H), 6.86 (dt, J=1.0, 6.8 Hz, 1H), 7.05-7.09 (m, 2H), 7.17 (dd, J=6.9, 9.2 Hz, 1H), 7.52 (s, 1H), 7.84-7.87 (m, 2H), 8.21 (d, J=9.2 Hz, 1H), 8.33 (d, J=6.9 Hz, 1H). 13C NMR (125 MHz, acetone-d₆) ppm 25.8, 26.7, 42.2, 57.1, 61.6, 67.9, 92.3, 94.2, 110.2, 114.0, 114.4, 116.5, 119.6, 124.6, 128.5, 129.7, 131.7, 135.9, 139.3, 139.4, 147.6, 156.0, 163.9.

(3,5-Dimethoxy-phenyl)-{3-[4-(2-isopropyl-indolizine-1-sulfonyl)-phenoxy]-propyl}-amine (1-50B). 2-Isopropyl-1-[[4-[(3-bromopropyl)oxy]-phenyl]sulfonyl]indolizine(8), 3,5-dimethoxyaniline (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

¹H NMR (500 MHz, CDCl₃) δ 1.23 (d, J=6.8 Hz, 6H), 3.32 (t, J=6.6 Hz, 2H), 3.55-3.64 (m, 1H), 3.74-3.77 (m, 6H), 4.09 (t, J=5.9 Hz, 2H), 5.81 (d, J=2.1 Hz, 1H), 5.85-5.90 (m, 1H), 6.72 (dt, J=1.1, 6.8 Hz, 1H), 6.89-6.92 (m, 2H), 7.04-7.08 (m, 1H), 7.14 (s, 1H), 7.83-7.86 (m, 2H), 7.91-7.94 (m, 1H), 8.26 (d, J=9.2 Hz, 1H). 13C NMR (125 MHz, CDCl₃) ppm 24.5, 24.9, 28.8, 40.9, 55.1, 55.2, 66.1, 89.8, 91.0, 91.7, 93.4, 107.8, 111.4, 112.6, 114.4, 118.3, 122.5, 125.6, 128.0, 134.4, 137.2, 138.1, 149.9, 161.6, 161.7.

[2-(3,4-Dimethoxy-phenyl)-ethyl]-{3-[4-(2-isopropyl-indolizine-1-sulfonyl)-phenoxy]-propyl}-amine (1-50C). 2-Isopropyl-1-[[4-[(3-bromopropyl)oxy]-phenyl]sulfonyl]indolizine(8), 2-(3,4-Dimethoxyphenyl)-ethylamine (2.5 eq, and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

¹H NMR (500 MHz, CDCl₃) δ 1.22 (d, J=6.9 Hz, 6H), 1.91-1.97 (m, 3H), 2.76 (t, J=7.0 Hz, 2H), 2.80 (t, J=6.9 Hz, 2H), 2.88 (t, J=7.0 Hz, 2H), 3.55-3.61 (m, 1H), 3.84 (s, 3H), 3.86 (s, 3H), 4.02 (t, J=6.1 Hz, 2H), 6.68-678 (m, 5H), 6.84-6.87 (m, 2H), 7.02-7.06 (m, 1H), 7.13 (s, 1H), 7.80-7.83 (m, 2H), 7.91 (d, J=6.8 Hz, 1H), 8.24 (d, J=9.2 Hz, 1H). 13C NMR (125 MHz, CDCl₃) ppm 24.4, 24.8, 29.3, 35.6, 46.5, 51.2, 55.8, 66.5, 107.8, 111.2, 111.3, 111.9, 112.5, 114.3, 118.2, 120.5, 122.4, 125.6, 127.9, 132.3, 134.3, 137.0, 138.0, 147.4, 148.9, 161.7.

Cyclohexyl-{3-[4-(2-isopropyl-indolizine-1-sulfonyl)-phenoxy]-propyl}-amine (1-52A). 2-Isopropyl-1-[[4-[(3-bromopropyl)oxy]-phenyl]sulfonyl]indolizine (8), cyclohexyl amine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

¹H NMR (500 MHz, CDCl₃) δ 1.22 (d, J=6.8 Hz, 6H), 1.61-1.71 (m, 3H), 1.81-1.86 (m, 2H), 2.24-2.29 (m, 2H), 2.46-2.52 (m, 1H), 3.03-3.10 (m, 1H), 3.15-3.23 (m, 1H), 3.53-3.60 (m, 1H), 4.09 (t, J=5.8 Hz, 2H), 6.72 (t, J=6.8 Hz, 1H), 6.86-6.90 (m, 2H), 7.04-7.09 (m, 1H), 7.13 (s, 1H), 7.81-7.84 (m, 2H), 7.93 (d, J=6.8 Hz, 1H), 8.24 (d, J=9.2 Hz, 1H), 9.02 (brs, 1H). 13C NMR (125 MHz, CDCl₃) ppm 24.5, 24.7, 24.9, 25.7, 29.1, 42.1, 57.9, 65.4, 107.6, 111.4, 112.7, 114.4, 118.3, 122.6, 125.7, 128.0, 134.4, 137.6, 138.1, 161.1.

1-{4-[3-(Azacyclododec-1-yl)-propoxy]-benzyl}-2-isopropyl-indolizine (1-76A). 2-Isopropyl-1-[[4-[(3-bromopropyl)oxy]-phenyl]sulfonyl]indolizine (8), dodecamethyleneimine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 8.16 (d, 1H, J=9.2 Hz), 7.82 (d, 1H, J=6.8 Hz), 7.74 (d, 2H, J=8.9 Hz), 7.03 (m, 1H), 6.95 (ddd, 1H, J=1.0 Hz, J=6.7 Hz, J=9.1 Hz), 6.80 (d, 2H, J=8.9 Hz), 6.61 (dt, 1H, J=1.2 Hz, J=6.8 Hz), 3.97 (t, 2H, J=6 Hz), 3.50 (sept., 1H, J=6.9 Hz), 2.38 (t, 2H, J=6.6 Hz), 2.24 (t, 4H, J=5.1 Hz), 1.78 (quint, 2H, J=6.4 Hz), 1.24 (m, 22H), 1.12 (d, 6H, J=6.9 Hz). 13-C NMR (125 MHz) 24.5, 24.8, 25.1, 25.5, 25.8, 26.1, 26.4, 27.0, 51.1, 55.4, 66.6, 108.2, 111.3, 112.5, 114.5, 118.4, 122.8, 126.2, 128.0, 134.4, 136.5, 137.8, 161.9.

tert-Butyl-{3-[4-(2-isopropyl-indolizin-1-ylmethyl)-phenoxy]-propyl}-amine (1-76D). 2-Isopropyl-1-[[4-[(3-bromopropyl)oxy]-phenyl]sulfonyl]indolizine (8), tert-butyl amine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 1.13 (d, 6H, J=6.8 Hz), 1.42 (s, 9H), 2.49 (quint, 2H, J=6.5 Hz), 3.06 (m, 2H), 3.48 (sept., 1H, J=6.8 Hz), 4.00 (t, 2H, J=5.8 Hz), 6.64 (dt, 1H, J=1.0 Hz, J=6.8 Hz), 6.80 (d, 2H, J=8.9 Hz), 6.98 (m, 1H), 7.05 (s, 1H), 7.74 (d, 2H, J=8.9 Hz), 7.84 (d, 1H, J=6.8 Hz), 8.15 (d, 1H, J=9.2 Hz), 8.88 (s, 1H). 13-C NMR (125 MHz) 24.5, 24.9, 26.0, 26.1, 39.5, 58.1, 65.5, 107.6, 111.4, 112.7, 114.4, 118.3, 122.6, 125.7, 128.0, 134.5, 137.6, 138.2, 161.2.

Dihexyl-{3-[4-(2-isopropyl-indolizin-1-ylmethyl)-phenoxy]-propyl}-amine (1-86A). 2-Isopropyl-1-[[4-[(3-bromopropyl)oxy]-phenyl]sulfonyl]indolizine (8), di-n-hexyl amine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 0.86 (t, 6H, J=6.8 Hz), 1.22 (d, 6H, J=6.9 Hz), 1.28 (m, 12H), 1.42 (m, 4H, J=5.6 Hz), 1.92 (m, 2H), 2.42 (m, 4H), 2.58 (m, 2H), 3.59 (sept., 1H, J=6.8 Hz), 4.03 (t, 2H, J=6.3 Hz), 6.71 (dt, 1H, J=1.1 Hz, J=6.8 Hz), 6.89 (d, 2H, J=8.9 Hz), 7.05 (ddd, 1H, J=0.9 Hz, J=6.7 Hz, J=9.1 Hz), 7.13 (s, 1H), 7.83 (d, 2H, J=8.9 Hz), 7.91 (d, 1H, J=6.9 Hz), 8.25 (d, 1H, J=9.1 Hz). 13-C NMR (125 MHz) 14.3, 22.9, 24.8, 25.1, 27.4, 32.0, 50.6, 54.4, 66.8, 108.2, 111.6, 112.8, 114.6, 118.6, 122.7, 125.9, 128.2, 134.6, 137.1, 138.4, 162.2.

2-Isopropyl-1-{4-[3-(4-phenyl-piperazin-1-yl)-propoxy]-benzyl}-indolizine (1-50E). 2-Isopropyl-1-[[4-[(3-bromopropyl)oxy]-phenyl]sulfonyl]indolizine (8), 4-phenylpiperidine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 1.23 (d, 6H, J=6.9 Hz), 2.04 (d, 3H, J=13.6 Hz), 2.44 (bs, 4H), 2.75 (bs, 3H), 3.10 (bs, 2H), 3.58 (m, 3H), 4.12 (t, 2H, J=5.7 Hz), 6.73 (dt, 1H, J=1.1 Hz, J=6.8 Hz), 6.89 (d, 2H, J=8.9 Hz), 7.07 (m, 1H), 7.14 (s, 1H), 7.30 (m, 6H), 7.85 (d, 2H, J=8.9 Hz), 7.93 (d, 1H, J=6.8 Hz), 8.25 (d, 1H, J=9.1 Hz). 13-C NMR (125 MHz) 24.8, 25.2, 111.7, 112.9, 114.6, 118.6, 122.9, 126.0, 127.1, 128.3, 129.1, 134.7, 138.4.

(3,4-Dimethoxy-phenyl)-{3-[4-(2-isopropyl-indolizin-1-ylmethyl)-phenoxy]-propyl}-amine (1-42A). 2-Isopropyl-1-[[4-[(3-bromopropyl)oxy]-phenyl]sulfonyl]indolizine (8), 3,4-Dimethoxyaniline (2.5 eq), and triethylamine (1.5 eq) were refluxed in toluene for 24h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 1.23 (d, 6H, J=6.8 Hz), 1.59 (bs, 1H), 2.10 (quint, 2H, J=6.3 Hz), 3.30 (t, 2H, J=6.6 Hz), 3.59 (td, 1H, J=6.8 Hz, J=13.7 Hz), 3.83 (s, 3H), 3.86 (s, 3H), 4.12 (t, 2H, J=5.9 Hz), 6.17 (dd, 1H, J=2.5 Hz, J=8.5 Hz), 6.26 (m, 3H), 6.34 (d, 1H, J=2.5 Hz), 6.73 (td, 4H, J=5.9 Hz, J=6.8 Hz), 6.92 (d, 2H, J=8.8 Hz), 7.06 (dd, 1H, J=6.8 Hz, J=9.1 Hz), 7.13 (s, 1H), 7.85 (d, 2H, J=8.8 Hz), 7.92 (d, 1H, J=6.8 Hz), 8.26 (d, 1H, J=9.2 Hz). 13-C NMR (125 MHz) 24.5, 24.9, 29.0, 41.8, 55.7, 56.6, 56.7, 66.2, 99.0, 100.7, 103.6, 106.4, 111.4, 112.6, 113.1, 113.3, 114.4, 118.4, 122.5, 125.6, 128.0, 134.4, 137.3, 138.2, 140.6, 141.7, 142.2, 142.9, 150.1, 161.6.

[2-(3,4-Dimethoxy-phenyl)-ethyl]-{3-[4-(2-isopropyl-indolizin-1-ylmethyl)-phenoxy]-propyl}-methyl-amine (1-38). 2-Isopropyl-1-[[4-[(3-bromopropyl)oxy]-phenyl]sulfonyl]indolizine (8), [2-(3,4-Dimethoxyphenyl)ethyl]-methyl amine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 1.21 (d, 6H, J=6.9 Hz), 1.96 (quint, 2H, J=6.7 Hz), 2.33 (s, 3H), 2.61 (m, 5H), 2.73 (dd, 3H, J=5.9 Hz, J=9.6 Hz), 3.58 (sept, 1H, J=6.8 Hz), 3.83 (s, 3H), 3.85 (s, 3H), 3.99 (t, 2H, J=6.3 Hz), 6.72 (m, 4H), 6.88 (d, 2H, J=8.9 Hz), 7.04 (ddd, 1H, J=0.9 Hz, J=6.7 Hz, J=9.1 Hz), 7.12 (s, 1H), 7.82 (d, 2H, J=8.9 Hz), 7.91 (d, 1H, J=6.7 Hz), 8.24 (d, 1H, J=9.1 Hz). 13-C NMR (125 MHz) 24.5, 24.8, 26.9, 33.2, 42.1, 53.8, 55.8, 55.9, 59.6, 66.3, 111.2, 111.3, 112.0, 112.6, 114.4, 118.3, 120.5, 122.4, 125.6, 127.9, 134.3, 136.9, 138.1, 147.3, 148.9, 161.8.

Dibutyl-{3-[4-(2-isopropyl-indolizin-1-ylmethyl)-phenoxy]-propyl}-amine (1-52C). 2-Isopropyl-1-[[4-[(3-bromopropyl)oxy]-phenyl]sulfonyl]indolizine (8), di-n-butyl amine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 0.89 (t, 6H, J=7.4 Hz), 1.21 (d, 6H, J=6.8 Hz), 1.31 (sext., 4H, J=7.5 Hz), 1.51 (m, 4H), 2.03 (m, 2H), 2.57 (bs, 4H), 2.74 (bs, 2H), 3.57 (sept., 1H, J=6.8 Hz), 4.04 (t, 2H, J=6.1 Hz), 6.71 (dt, 1H, J=0.7 Hz, J=6.8 Hz), 6.88 (d, 2H, J=8.8 Hz), 7.05 (dd, 1H, J=6.8 Hz, J=9.1 Hz), 7.13 (s, 1H), 7.82 (d, 2H, J=8.8 Hz), 7.92 (d, 1H, J=6.9 Hz), 8.24 (d, 1H, J=9.2 Hz). 13-C NMR (125 MHz) 13.9, 20.5, 24.5, 24.8, 50.3, 53.5, 66.1, 107.8, 111.3, 112.6, 114.3, 118.3, 122.4, 125.6, 127.9, 134.3, 137.0, 138.0, 161.6.

### Toluene-4-sulfonic acid 4-(2-isopropyl-indolizine-1-sulfonyl)-phenyl ester (SrOTs) (6)

1H-NMR (500 MHz) ppm 1.20 (d, 6H, J=6.9 Hz), 2.47 (s, 3H), 3.51 (sept., 1H, J=6.8 Hz), 6.77 (dt, 1H, J=1.2 Hz, J=6.8 Hz), 7.09 (m, 4H), 7.32 (d, 2H, J=8.0 Hz), 7.68 (d, 2H, J=8.3 Hz), 7.84 (d, 2H, J=8.9 Hz), 7.96 (d, 1H, J=6.8 Hz), 8.23 (d, 1H, J=9.2 Hz). 13-C NMR (125 MHz) 21.7, 24.4, 24.9, 111.8, 113.0, 118.2, 122.8, 123.1, 125.9, 127.7, 128.5, 129.9, 131.8, 134.9, 138.4, 143.8, 145.8, 151.9.

2-Isopropyl-1-{4-[3-(4-phenyl-piperazin-1-yl)-propoxy]-benzenesulfonyl}-indolizine (1-50D). 2-Isopropyl-1-[[4-[(3-bromopropyl)oxy]-phenyl]sulfonyl]indolizine (8), 1-phenylpiperazine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 1.23 (d, 6H, J=6.9 Hz), 1.98 (quint, 2H, J=6.6 Hz), 2.07 (td, 2H, J=2.2 Hz, J=4.4 Hz), 2.53 (t, 2H, J=7.0 Hz), 2.57 (m, 4H), 2.87 (bs, 1H), 3.16 (m, 4H), 3.62 (sept., 1H, J=6.9 Hz), 4.15 (t, 2H, J=6.4 Hz), 6.79 (t, 1H, J=7.3 Hz), 6.84 (dt, 1H, J=1.1 Hz, J=6.8 Hz), 6.94 (d, 2H, J=8.0 Hz), 7.06 (d, 2H, J=8.9 Hz), 7.16 (ddd, 1H, J=0.9 Hz, J=6.8 Hz, J=9.1 Hz), 7.22 (dd, 2H, J=7.4 Hz, J=8.6 Hz), 7.50 (s, 1H), 7.86 (d, 2H, J=8.9 Hz), 8.22 (d, 1H, J=9.2 Hz), 8.31 (d, 1H, J=6.9 Hz). 13-C NMR (125 MHz) 25.8, 26.6, 28.3, 50.7, 55.0, 56.3, 68.3, 110.2, 113.9, 114.4, 116.4, 117.5, 119.6, 120.8, 124.5, 128.4, 129.7, 130.7, 135.9, 139.3, 153.5, 164.0.

{3-[4-(2-Isopropyl-indolizine-1-sulfonyl)-phenoxy]-propyl}-(3,4,5-trimethoxy-benzyl)-amine (1-42B). 2-Isopropyl-1-[[4-[(3-bromopropyl)oxy]-phenyl]sulfonyl]indolizine (8), 3,4,5-Trimethoxybenzylamine (2.5 eq), and triethylamine (1.5 eq) were refluxed in toluene for 24 h. The solvent was then eliminated under vacuum and the residue was taken up in H₂O. The medium was extracted with CH₂Cl₂, dried over Na₂SO₄ and evaporated to dryness. The oil obtained was purified via flash chromatography using CH₂Cl₂/MeOH as the eluent.

1H-NMR (500 MHz) ppm 1.13 (d, 6H, J=6.9 Hz), 1.18 (s, 1H), 1.92 (m, 4H), 2.75 (t, 2H, J=6.9 Hz), 3.39 (s, 1H), 3.49 (sept., 1H, J=6.9 Hz), 3.67 (s, 2H), 3.74 (s, 9H), 3.99 (t, 2H, J=6.1 Hz), 6.48 (s, 2H), 6.62 (dt, 1H, J=1.1 Hz, J=6.8 Hz), 6.80 (d, 2H, J=8.9 Hz), 6.96 (ddd, 1H, J=0.9 Hz, J=6.7 Hz, J=9.1 Hz), 7.04 (s, 1H), 7.74 (d, 2H, J=8.9 Hz), 7.83 (d, 1H, J=6.8 Hz), 8.15 (d, 1H, J=9.2 Hz). 13-C NMR (125 MHz) 24.8, 29.2, 29.7, 54.1, 60.7, 66.5, 105.0, 111.4, 112.6, 114.4, 118.3, 122.5, 125.7, 127.9, 134.3, 137.0, 138.1, 153.2, 161.7.

### Toluene-4-sulfonic acid 4-(pyridin-2-ylmethanesulfonyl)-phenyl ester (5)

1H-NMR (500 MHz) ppm 2.48 (s, 3H), 4.55 (s, 2H), 7.10 (d, 2H, J=8.7 Hz), 7.26 (m, 1H), 7.35 (d, 2H, J=8.1 Hz), 7.46 (d, 2H, J=7.8 Hz), 7.61 (d, 2H, J=8.7 Hz), 7.70 (d, 3H, J=8.1 Hz), 8.40 (d, 1H, J=4.2 Hz). 13-C NMR (125 MHz) 21.6, 64.6, 122.9, 123.5, 125.8, 128.5, 130.0, 130.4, 131.8, 136.8, 136.9, 140.0, 148.6, 149.7, 153.4.

### 1-[4-(3-Bromo-propoxy)-benzenesulfonyl]-2-isopropyl-indolizine (8)

1H-NMR (500 MHz) ppm 1.24 (d, 6H, J=6.8 Hz), 2.32 (quint, 2H, J=6.1 Hz), 3.60 (m, 3H), 4.13 (t, 2H, J=5.8 Hz), 6.73 (dd, 1H, J=3.8 Hz, J=9.8 Hz), 6.92 (d, 2H, J=8.9 Hz), 7.06 (m, 1H), 7.14 (s, 1H), 7.86 (d, 2H, J=8.8 Hz), 7.93 (d, 1H, J=6.9 Hz), 8.26 (d, 1H, J=9.2 Hz). 13-C NMR (125 MHz) 24.8, 25.1, 29.8, 32.3, 65.8, 108.1, 111.6, 112.9, 114.7, 118.6, 122.7, 125.9, 128.3, 134.7, 137.6, 138.4, 161.7.

Toluene-4-sulfonic acid 4-[4-(quinolin-2-ylmethanesulfonyl)-phenoxy]-phenyl ester: To a solution of NaHCO₃ (4.9 g, 58.38 mmol, 5 equiv.) and Na₂SO₃ (2.94 g, 23.35 mmol, 2 equiv.) in H₂O (20 mL) was slowly added the sulfonyl chloride (by portion and the reaction was stirred at rt for 2 hours. Then the pycolyl hydrochloric salt (4.04 g, 11.7 mmol, 1 equiv.) was added very slowly by portion and the reaction was stirred 10 minutes at rt and then warmed up to 100 C for 5 hours. The reaction was cooled to rt and extracted with DCM several times. The crude product was used for the next step without further purification. White solid (49% Yld; 3.10 g; 5.68 mmol); R_{f} 0.5 (EtOAC/Hexanes 2:1); ¹H NMR (CDCl₃, 500 MHz) ppm 2.45 (3H, s, CH₃), 4.74 (2H, s, CH₂), 7.03 (2H, d, J=8.8 Hz, CH_{Ar}), 7.28 (2H, d, J=8.3 Hz, CH_{Ar}), 7.60 (4H, d, J=8.8 Hz, CH_{Ar}), 7.65 (2H, d, J=8.3 Hz, CH_{Ar}), 7.76 (H, t, J=8.3 Hz, CH_{Ar}), 7.82 (1H, d, J=8.3 Hz, CH_{Ar}), 7.85 (1H, d, J=8.88 Hz, CH_{Ar}), 8.20 (1H, d, J=8.3 Hz, CH_{Ar}).

### Example 3

### In Vivo Studies

An acute in vivo study was conducted with mice. Mice (Tg PS1/APPsw; 4-month-old) were injected intraperitoneally with 10 mg/Kg of the compound for 4 days or the vehicle only (100 microL of DMSO). One hour after the last injection, mice were euthanatized, their brains and plasma collected. Aβ1-40 and Aβ1-42 were evaluated in the plasma and brain water soluble Aβ1-40 and Aβ1-42 were extracted and quantified by ELISAs (Biosource, Calif.). Protein concentrations were determined in the different samples using the BCA method and results were calculated in pg of Aβ per mg of protein. Results were finally expressed as a % of the values calculated in animals receiving the vehicle only (% of control).

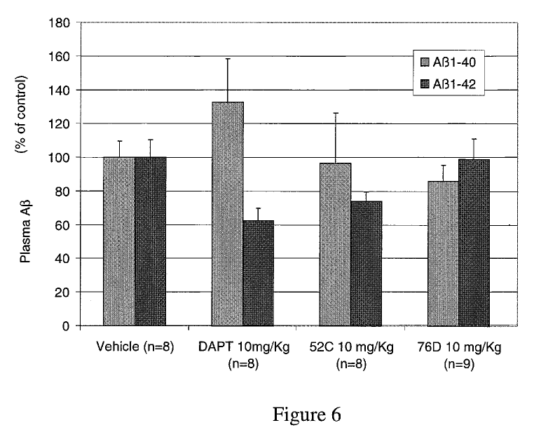

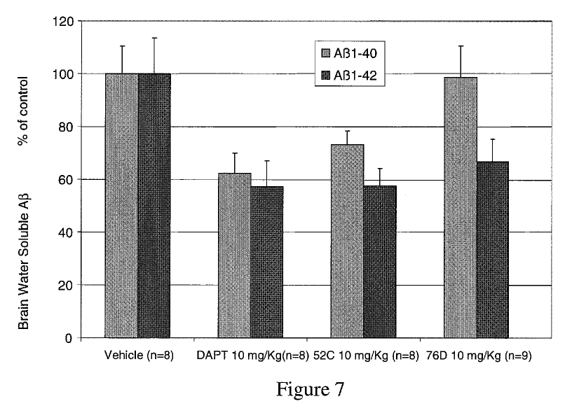

The results with compounds 1-52C and 1-76D are shown in FIG. 6 below which shows plasma beta-amyloid (Aβ) (% of control) using 10 mg/Kg compound. FIG. 7 shows brain water soluble beta-amyloid (Aβ) (% of control) using 10 mg/Kg of the compounds 1-52C and 1-76D. The control was vehicle. Also shown are the results with DAPT (1,3-Diazido-2-propyl ester of 4,4,4-trinitrobutyric acid), a known inhibitor of beta-amyloid.

What is claimed is:

1. A method for treating a disease associated with cerebral accumulation of Alzheimer's amyloid, said method comprising administering to an animal or human in need thereof a therapeutically effective amount of a compound of Formula I, II, III or IV, or a salt, ester, or prodrug thereof, wherein the compound optionally reduces β-amyloid production by at least about 5%, 10%, 15%, 20%, 25%, 30%, or 50%, in cells that overexpress APP or a fragment thereof, wherein the compound of Formula I is: 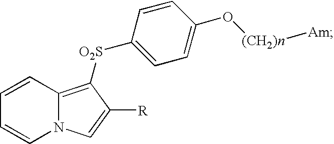
wherein the compound of Formula II is: 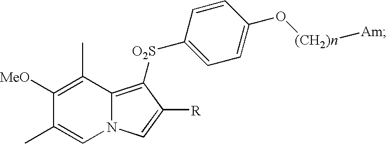
wherein the compound of Formula III is: 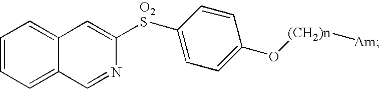
and
wherein the compound of Formula IV is: 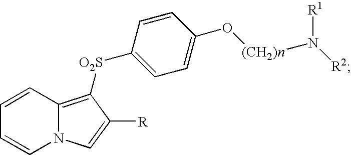
wherein for Formulas I, II, and III,
n is 3, 4 or 5;
R is optionally substituted alkyl, e.g., methyl, ethyl, isopropyl, butyl, isobutyl; optionally substituted alkenyl; optionally substituted alkynyl; optionally substituted cycloalkyl; optionally substituted heteroalkyl; optionally substituted aryl; optionally substituted heteroaryl; or acyl; or any of the groups listed for R in Table 1;
Am is a residue of an amine, such as an optionally substituted alkyl amine, or Am is optionally selected from the groups listed in Table 2 or 3; and
wherein for Formula IV,
n is 1, 2 or 3;
R is optionally substituted alkyl, optionally substituted alkenyl or optionally substituted alkynyl, or in one embodiment, a C₁₋₁₀ alkyl, e.g. methyl, ethyl, propyl, butyl, secbutyl, isopropyl, or isobutyl;
R¹ is H, unsubstituted alkyl or substituted alkyl, such as C1, C2, C3, C4, C5, C6, C7, C8, C9, C10 unsubstituted or substituted alkyl, including straight chain, branched or cycloalkyl or is the same as R²;
R² is unsubstituted alkyl or substituted alkyl, such as C1, C2, C3, C4, C5, C6, C7, C8, C9, C10 unsubstituted or substituted alkyl, including straight chain, branched or cycloalkyl, wherein the substituent is, e.g., a substituted or unsubstituted aromatic group; and optionally R¹ and R² together form an optionally substituted heterocycle, such as an optionally substituted 5, 6, 7, 8, 9, 10, 11, 12, 13 or 14 membered ring saturated or unsaturated heterocycle, including one or more heteroatoms such as nitrogen.

2. A method according to claim 1, wherein for Formula I, II, or III, R is selected from the group consisting of Table 1, and Am is selected from the group consisting of Table 2 or Table 3;
and
wherein for Formula IV,
n is 1, 2 or 3;
R is a C₁₋₁₀ alkyl;
R¹ is a C1, C2, C3, C4, C5, C6, C7, C8, C9, or C10 unsubstituted or substituted alkyl;
R² is a C1, C2, C3, C4, C5, C6, C7, C8, C9, C10 unsubstituted or substituted alkyl, wherein the substituent for R² if present is a substituted or unsubstituted aromatic group.

3. A method according to any of claims 1-2, wherein for Formula I, II, or III, R is selected from H, methyl, ethyl, isopropyl, butyl, and isobutyl, and Am is an alkyl amine;
and
wherein for Formula IV, R is selected from the group consisting of methyl, ethyl, propyl, butyl, secbutyl, isopropyl, or isobutyl; R¹ is a C1, C2, C3, C4, C5, C6, C7, C8, C9, or C10 unsubstituted alkyl; and R² is a C1, C2, C3, C4, C5, C6, C7, C8, C9, C10 unsubstituted alkyl.

4. A method according to any of claims 1-3, wherein the compound is selected from the group consisting of

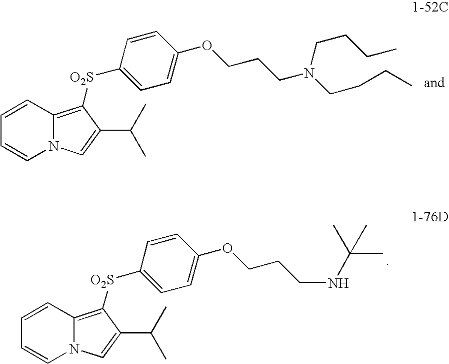

5. A method according to any of claims 1-4, wherein the therapeutically effective amount of the compound is between about 0.02 to 1000 mg per unit dose; or about 0.5 to 500 mg per unit dose.

6. A method according to any of claims 1-5, wherein the route of administration of the compound to the animal or human is parenteral, oral or intraperitoneal.

7. A method according to any of claims 1-6, wherein the compound is administered orally in a unit dosage form selected from the group consisting of hard or soft shell gelatin capsules, tablets, troches, sachets, lozenges, elixirs, suspensions, syrups, wafers, powders, granules, solutions and emulsions.

8. A method according to any of claims 1-7, wherein the method comprises administration to a human in need thereof.

9. A method according to any of claims 1-8, wherein the disease is selected from the group consisting of traumatic brain injury, Alzheimer's disease, cerebral amyloid angiopathy, hereditary cerebral hemorrhage with amyloidosis Dutch-type, or other forms of familial AD and familial cerebral Alzheimer's amyloid angiopathy.

10. A method according to any of claims 1-9, wherein the disease is Alzheimer's disease.

11. A compound of Formula I, II, III or IV, or a salt, ester, or prodrug thereof, wherein the compound of Formula I is: 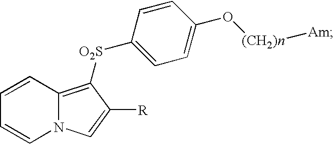
wherein the compound of Formula II is: 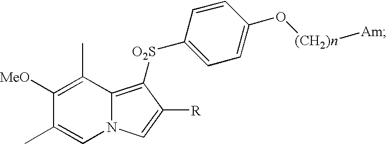
wherein the compound of Formula III is: 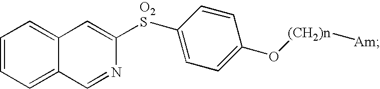
and
wherein the compound of Formula IV is: 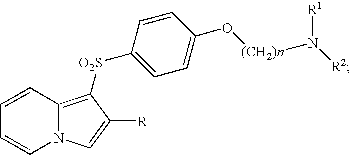
wherein for Formulas I, II, and III,
n is 3, 4 or 5;
R is optionally substituted alkyl, e.g., methyl, ethyl, isopropyl, butyl, isobutyl; optionally substituted alkenyl; optionally substituted alkynyl; optionally substituted cycloalkyl; optionally substituted heteroalkyl; optionally substituted aryl; optionally substituted heteroaryl; or acyl; or any of the groups listed for R in Table 1;
Am is a residue of an amine, such as an optionally substituted alkyl amine, or Am is optionally selected from the groups listed in Table 2 or 3;
and
wherein for Formula IV,
n is 1, 2 or 3;
R is optionally substituted alkyl, optionally substituted alkenyl or optionally substituted alkynyl, or in one embodiment, a C1-10 alkyl, e.g. methyl, ethyl, propyl, butyl, secbutyl, isopropyl, or isobutyl;
R¹ is H, unsubstituted alkyl or substituted alkyl, such as C1, C2, C3, C4, C5, C6, C7, C8, C9, C10 unsubstituted or substituted alkyl, including straight chain, branched or cycloalkyl or is the same as R²;
R² is unsubstituted alkyl or substituted alkyl, such as C1, C2, C3, C4, C5, C6, C7, C8, C9, C10 unsubstituted or substituted alkyl, including straight chain, branched or cycloalkyl, wherein the substituent is, e.g., a substituted or unsubstituted aromatic group;
and optionally R¹ and R² together form an optionally substituted heterocycle, such as an optionally substituted 5, 6, 7, 8, 9, 10, 11, 12, 13 or 14 membered ring saturated or unsaturated heterocycle, including one or more heteroatoms such as nitrogen.

12. A compound according to claim 11, wherein for Formula I, II, or III, R is selected from the group consisting of Table 1, and Am is selected from the group consisting of Table 2 or Table 3;
and
wherein for Formula IV,
n is 1, 2 or 3;
R is a C₁₋₁₀ alkyl;
R¹ is a C1, C2, C3, C4, C5, C6, C7, C8, C9, or C10 unsubstituted or substituted alkyl;
R² is a C1, C2, C3, C4, C5, C6, C7, C8, C9, C10 unsubstituted or substituted alkyl, wherein the substituent for R² if present is a substituted or unsubstituted aromatic group.

13. A compound according to claim 11, wherein for Formula I, II, or III, R is selected from H, methyl, ethyl, isopropyl, butyl, and isobutyl, and Am is an alkyl amine;
and
wherein for Formula IV, R is selected from the group consisting of methyl, ethyl, propyl, butyl, secbutyl, isopropyl, or isobutyl; R¹ is a C1, C2, C3, C4, C5, C6, C7, C8, C9, or C10 unsubstituted alkyl; and R² is a C1, C2, C3, C4, C5, C6, C7, C8, C9, C10 unsubstituted alkyl.

14. A compound according to claim 11, wherein the compound is selected from the group consisting of

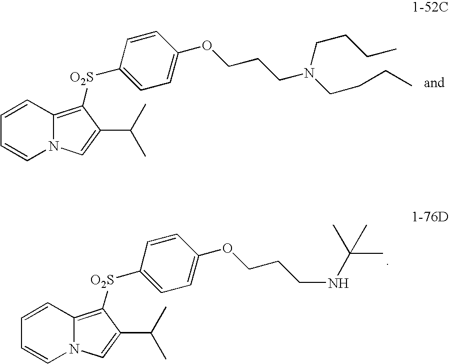

15. A pharmaceutical composition comprising a therapeutically effective amount of a compound according to any of claims 11 through 14 or a salt, ester, or prodrug thereof, and a pharmaceutically acceptable carrier.
